(19) United States
(12) Patent Application Publication
(10) Pub. No.: US 2024/0285514 A1
(43) Pub. Date: Aug. 29, 2024

(54) EXTRACTS OF EUTERPE OLERACEA, METHODS OF MAKING, AND USES THEREOF

(71) Applicant: Naturesphix Limited, London (GB)

(72) Inventors: João Batista CALIXTO, Florianópolis (BR); Melina HELLER, Florianópolis (BR); Camila Guimarães Moreira ZIMMER, Florianópolis (BR); Jarbas Mota SIQUEIRA JUNIOR, Florianópolis (BR); Cristina Setim FREITAS, Florianópolis (BR); Rodrigo MARCON, Florianópolis (BR)

(21) Appl. No.: 18/570,454

(22) PCT Filed: Jun. 9, 2022
(86) PCT No.: PCT/GB2022/051448

§ 371 Date: Dec. 14, 2023

Related U.S. Application Data
(60) Provisional application No. 63/210,332, filed on Jun. 14, 2021.

(51) Int. Cl. A61K 8/9794; A61K 36/889; A61Q 17/04; A61Q 19/08

(52) U.S. Cl. CPC A61K 8/9794; A61K 36/889; A61Q 17/04; A61Q 19/08; A61K 2236/333; A61K 2236/51; A61K 2800/522

(57) ABSTRACT

Described herein are extracts and compositions of Euterpe oleracea, methods of preparation, and methods of use. The extracts and compositions can be used in various forms of skin care, including as an antioxidant, photoprotector, for soothing skin discomfort, and to reduce or prevent the effects of aging on the skin.

### CROSS REFERENCE TO RELATED APPLICATIONS

The present application claims priority to U.S. Provisional Application No. 63/210,332, filed on Jun. 14, 2021, the disclosure of which is incorporated by reference herein in its entirety.

### BACKGROUND

The plant Euterpe oleracea contains in its fruit and seeds a number of compounds which can be health promoting. These compounds include a variety of antioxidants, which may treat or prevent oxidative damage to a variety of human tissues, among other uses. Administration of the beneficial compounds of Euterpe species can be facilitated by the preparation of extracts of the plant matter. One method of preparing Euterpe extracts is reported in European patent EP 2051721B. There is, however, a need for alternative methods of preparing Euterpe species extracts with effective levels of beneficial phytochemical constituents, for use in treating or preventing human disease, including damage to human skin and for use in cosmetic applications or otherwise promoting human health.

### SUMMARY

In one aspect, provided herein is a method of preparing an Euterpe oleracea extract, the method comprising:
(a) contacting Euterpe oleracea material with a first solvent at a first temperature below boiling to produce a first wet mixture, and then milling the first wet mixture to obtain a second mixture; or milling Euterpe oleracea material to produce a first dry mixture, and then contacting the first dry mixture with a first solvent at a first temperature below boiling to obtain a second mixture;
(b) contacting the second mixture with an extractor liquid to obtain a crude extract;
(c) filtering the crude extract to obtain a purified extract; and
(d) drying the purified extract to produce the Euterpe oleracea extract using a drying process selected from the group consisting of lyophilization and spray drying;
wherein there is no more than one step that includes heating to at least the boiling point at about one atmosphere, other than the spray drying step.

In some embodiments, provided herein is a Euterpe oleracea extract prepared according to methods described herein, the extract comprising:
about 0.5 μg/g to about 50 μg/g vanillic acid;
about 5 μg/g to about 100 μg/g compound of unknown identity, with the same molecular mass of vanillic acid;
about 2 μg/g to about 120 μg/g ferulic acid;
about 5 μg/g to about 200 μg/g compound of unknown identity with the same molecular mass of ferulic acid;
about 0 μg/g to about 250 μg/g p-coumaric acid;
about 2 μg/g to about 80 μg/g hispidulin;
about 10 μg/g to about 70 μg/g rutin;
about 0 mg/g to about 20 mg/g procyanidin;
about 0.5 mg/g to about 30 mg/g epicatechin and catechin;
about 0 μg/g to about 25 μg/g naringenin; and
about 0 μg/g to about 10 μg/g quercetin.
wherein the extract is stable for about 16 weeks at a temperature of from about 20 to about 30° C. as measured by HPLC.

In some embodiments, provided herein is a Euterpe oleracea extract prepared according to methods described herein, the extract comprising:
about 5 μg/g to about 50 μg/g vanillic acid;
about 50 μg/g to about 100 μg/g compound of unknown identity, with the same molecular mass of vanillic acid;
about 10 μg/g to about 50 μg/g ferulic acid;
about 100 μg/g to about 200 μg/g compound of unknown identity with the same molecular mass of ferulic acid;
about 100 μg/g to about 250 μg/g p-coumaric acid;
about 10 μg/g to about 50 μg/g hispidulin;
about 30 μg/g to about 60 μg/g rutin;
about 10 mg/g to about 20 mg/g procyanidin;
about 15 mg/g to about 30 mg/g epicatechin and catechin;
about 5 μg/g to about 15 μg/g naringenin; and
about 0 μg/g to about 10 μg/g quercetin.
wherein the extract is stable for about 16 weeks at a temperature of from about 20 to about 30° C. as measured by HPLC.

In some embodiments, provided herein is a Euterpe oleracea extract prepared according to methods described herein, the extract comprising:
about 0.5 μg/g to about 2 μg/g vanillic acid;
about 2 μg/g to about 6 μg/g compound of unknown identity, with the same molecular mass of vanillic acid;
about 5 μg/g to about 50 μg/g ferulic acid;
about 100 μg/g to about 40 μg/g compound of unknown identity with the same molecular mass of ferulic acid;
about 0 μg/g to about 1 μg/g p-coumaric acid;
about 2 μg/g to about 20 μg/g hispidulin;
about 10 μg/g to about 70 μg/g rutin;
about 0 mg/g to about 20 mg/g procyanidin;
about 100 μg/g to about 1000 μg/g epicatechin and catechin;
about 5 μg/g to about 15 μg/g naringenin; and about 0 μg/g to about 10 μg/g quercetin.
wherein the extract is stable for about 16 weeks at a temperature of from about 20 to about 30° C. as measured by HPLC.

In another aspect, provided herein is a method of treating the skin of a subject, the method comprising contacting the skin with an effective amount of a Euterpe oleracea extract produced according to methods described herein.

### BRIEF DESCRIPTION OF THE FIGURES

FIG. 1 is an overlay of HPLC traces of several components of Euterpe extracts (plot of intensity vs. retention time in min.).

FIG. 2 is a plot of antioxidant activity (%) vs. concentration of TI-35 (μg/mL).

FIG. 3 is a plot of antioxidant activity (%) vs. concentration of ascorbic acid (μg/mL).

FIG. 4 is a plot of keratinocyte viability (%) vs. concentration of TI-35 after 24 hours (μg/mL).

FIG. 5 is a plot of keratinocyte viability (%) vs. concentration of TI-35 after 48 hours (μg/mL).

FIG. 6 is a plot of keratinocyte viability (%) vs. concentration of TI-35 after 96 hours (μg/mL).

FIG. 7 is a plot of keratinocyte proliferation (% vehicle) vs. concentration of TI-35 after 96 hours (μg/mL).

FIG. 8 is a plot of ROS production (% vehicle) vs. concentration of various agents (μg/mL).

FIG. 9 is a plot of ROS production (% vehicle+UVB) vs. concentration of various agents (μg/mL) to determine scavenger potential in keratinocytes.

FIG. 10 is a plot of ROS production (% vehicle+UVB) vs. concentration of various agents (μg/mL) to determine antioxidant potential in keratinocytes.

FIG. 11 is a plot of MMP-1 activity (% vehicle) vs. concentration of various agents (μg/mL).

FIG. 12 is a plot of fibroblast viability (% vehicle) vs. concentration of TI-35 (μg/mL) at 24 hours.

FIG. 13 is a plot of fibroblast viability (% vehicle) vs. concentration of TI-35 (μg/mL) at 48 hours.

FIG. 14 is a plot of ROS production (% vehicle+UVB) vs. concentration of various agents (μg/mL) to determine scavenger potential in fibroblasts.

FIG. 15 is a plot of ROS production (% vehicle+UVB) vs. concentration of various agents (μg/mL) to determine antioxidant potential in fibroblasts.

FIG. 16 is a plot of tyrosinase activity (% vehicle) vs. concentration of TI-35 (μg/mL).

FIG. 17 is a plot of tyrosinase activity (% vehicle) vs. concentration of kojic acid (μg/mL).

FIG. 18 is a plot of cellular viability (% vehicle) vs. concentration of ST-035 (μg/mL).

FIG. 19 is a plot of cellular viability (% vehicle) vs. concentration of kojic acid (μg/mL).

FIG. 20 is a plot of melanin production (μg/mL) for vehicle and TI-35.

FIG. 21 is a plot of skin irritation (% PBS) for SDS and TI-35.

FIG. 22-24 are charts of mutagenicity of TI-35 in the absence of S9 activation.

FIGS. 25A and 25B are charts of mutagenicity of TI-35 in the presence of S9 activation.

### DETAILED DESCRIPTION

In one aspect, provided herein is a method of preparing an Euterpe oleracea extract, the method comprising:
(a) contacting Euterpe oleracea material with a first solvent at a first temperature below boiling to produce a first wet mixture, and then milling the first wet mixture to obtain a second mixture; or milling Euterpe oleracea material to produce a first dry mixture, and then contacting the first dry mixture with a first solvent at a first temperature below boiling to obtain a second mixture;
(b) contacting the second mixture with an extractor liquid to obtain a crude extract;
(c) filtering the crude extract to obtain a purified extract; and
(d) drying the purified extract to produce the Euterpe oleracea extract using a drying process selected from the group consisting of lyophilization and spray drying;
wherein there is no more than one step that includes heating to at least the boiling point at about one atmosphere, other than the spray drying step.

In some embodiments, provided here in is a method of preparing an Euterpe oleracea extract, the method comprising:
(a) milling Euterpe oleracea material to produce a first dry mixture, and then contacting the first dry mixture with a first solvent at a first temperature below boiling to obtain a second mixture;
(b) contacting the second mixture with an extractor liquid to obtain a crude extract;
(c) filtering the crude extract to obtain a purified extract; and
(d) drying the purified extract to produce the Euterpe oleracea extract using spray drying;
wherein there is no more than one step that includes heating to at least the boiling point at about one atmosphere, other than the spray drying step.

In one aspect, provided herein is a method of preparing an Euterpe oleracea extract, the method comprising:
(a) contacting Euterpe oleracea material with a first solvent at a first temperature below boiling to produce a first wet mixture, and milling the first wet mixture to obtain a second mixture;
(b) contacting the second mixture with an extractor liquid to obtain a crude extract;
(c) filtering the crude extract to obtain a purified extract; and
(d) lyophilizing the purified extract to produce the Euterpe oleracea extract;
wherein there is no more than one step that includes heating at least the to boiling point at about one atmosphere.

In some embodiments, in step (a), the first solvent is water and the first temperature is about 20° C. to about 30° C.

In some embodiments, the Euterpe oleracea material is washed in water and dried at a temperature between about 20° C. to about 30° C. prior to step (a).

In some embodiments, the method further comprises wherein in step (a) the second mixture is heated to boiling after milling.

In some embodiments, there is no step that includes heating to at least the boiling point at one atmosphere.

In some embodiments, there is no step that includes heating at a temperature greater than 60° C., greater than 50° C., greater than 40° C., or greater than 30° C.

In some embodiments, during or after milling the first mixture, the milled first mixture is passed through a mesh of 4-16 mm, or about 8 mm, to obtain the second mixture.

In some embodiments, in step (b), the extractor liquid comprises ethanol.

In some embodiments, the method comprises maintaining the second mixture contacted with an extractor liquid at a temperature of about 1° C. to about 5° C. for a period of about 1 day to about 10 days.

In some embodiments, the method comprises maintaining the second mixture contacted with an extractor liquid at a temperature of about 1° C. to about 5° C. for a period of about 1 day to about 7 days.

In some embodiments, the method comprises maintaining the second mixture contacted with an extractor liquid at a temperature of about 1° C. to about 5° C. for a period of about 1 day to about 3 days.

In some embodiments, step (b) is performed in a reactor for less than 24 hours.

In some embodiments, step (b) is performed in a reactor for less than 4 hours, less than 3 hours, less than 2 hours, or less than 1 hour.

In some embodiments, step (b) is performed under substantially constant agitation and at a temperature of 30-60° C., 35-55° C., about 40° C., or about 50° C.

In some embodiments, in step (b), the ratio of particulate material to the extractor liquid is about 1:6 to about 6:1 weight/volume.

In some embodiments, in step (b), the ratio of particulate material to the extractor liquid is about 1:4 to about 4:1 weight/volume.

In some embodiments, in step (b) the ratio of particulate material to the extractor liquid is from about 1:2 to about 1:10, or about 1:4 weight/volume.

In some embodiments, the extractor liquid comprises ethanol and the first solvent comprises water.

In some embodiments, the percent (v/v) of ethanol to water in step (b) is 75% ethanol and 25% water.

In some embodiments, the percent (v/v) of ethanol to water in step (b) is 85% ethanol and 15% water.

In some embodiments, the ratio of ethanol to water (v/v) in step (b) is about 0.1:1 to about 1:1.

In some embodiments, in step (b), the ratio of ethanol to water (v/v) is about 1:1.

In some embodiments, in step (c), filtering the crude extract comprises
(i) passing the crude extract through a first filter, wherein the first filter is comprised of a voile-type fabric; and
(ii) passing the crude extract through a second filter, wherein the second filter has a basis weight of about 50-80.0 g/m².

In some embodiments, the first filter comprises paper.

In some embodiments, the second filter comprises paper of weight from about 50 g/m² to about 80 g/m².

In some embodiments, the purified extract is pasteurized before step (d). In some embodiments, the pasteurization is performed by heating to a temperature below the boiling point of the purified extract.

In some embodiments, in step (d), lyophilizing the purified extract comprises
(i) removing the extractor liquid from the purified extract via rotary evaporation to obtain a concentrated extract; and
(ii) lyophilizing the concentrated extract to produce the Euterpe oleracea extract.

In some embodiments, removing the extractor liquid from the crude extract via rotary evaporation is conducted at a temperature of about 30° C. to about 70° C.

In some embodiments, lyophilization of the concentrated extract is conducted at a temperature of about −35° C. to about −55° C. and a pressure of about 200 μm Hg to about 300 μm Hg.

In some embodiments, the method further comprises the steps of heating the second mixture to about 90 to 110° C.; and cooling the second mixture to a temperature between about 20° C. to about 30° C. after contacting the second mixture with an extractor liquid and prior to filtering the crude extract.

In some embodiments, heating the second mixture to 90 to 110° C. further comprises holding the second mixture at 90 to 110° C. for about 10 minutes.

In some embodiments, the second mixture is cooled to a temperature of about 20° C.

In some embodiments, in step (d), spray drying the purified extract comprises
(i) removing the extractor liquid from the purified extract via rotary evaporation to obtain a concentrated purified extract; and
(ii) spray drying the concentrated purified extract to produce the Euterpe oleracea extract.

In some embodiments, excipients are added to the concentrated purified extract before spray drying in an amount of 40-60% w/v based on the volume of the concentrated purified extract prior to spray drying.

In some embodiments, the excipients comprise maltodextrin and silicon dioxide. In some embodiments, the excipients comprise 1-4% w/v silicon dioxide and 40-60% w/v maltodextrin, based on the volume of the concentrated purified extract prior to spray drying.

In some embodiments, the final temperature during spray drying is 85-105° C.

In some embodiments, the Euterpe oleracea extract has a maximum water content of 6%.

In some embodiments, the Euterpe oleracea extract comprises one or more phenolic compounds selected from the group consisting of vanillic acid, isovanillic acid, ferulic acid, isoferulic acid, hispidulin, rutin, naringenin, quercetin, p-coumaric acid, o-coumaric acid, a procyanidin, epicatechin, catechin, and combinations thereof.

In some embodiments, provided herein is a Euterpe oleracea extract prepared according to methods described herein, the extract comprising:
about 0.5 μg/g to about 50 μg/g vanillic acid;
about 5 μg/g to about 100 μg/g compound of unknown identity, with the same molecular mass of vanillic acid;
about 2 μg/g to about 120 μg/g ferulic acid;
about 5 μg/g to about 200 μg/g compound of unknown identity with the same molecular mass of ferulic acid;
about 0 μg/g to about 250 μg/g p-coumaric acid;
about 2 μg/g to about 80 μg/g hispidulin;
about 10 μg/g to about 70 μg/g rutin;
about 0 mg/g to about 20 mg/g procyanidin;
about 0.5 mg/g to about 30 mg/g epicatechin and catechin;
about 0 μg/g to about 25 μg/g naringenin; and about 0 μg/g to about 10 μg/g quercetin.
wherein the extract is stable for about 16 weeks at a temperature of from about 20 to about 30° C. as measured by HPLC.

In some embodiments, provided herein is a Euterpe oleracea extract prepared according to methods described herein, the extract comprising:
about 5 μg/g to about 50 μg/g vanillic acid;
about 50 μg/g to about 100 μg/g compound of unknown identity, with the same molecular mass of vanillic acid;
about 10 μg/g to about 50 μg/g ferulic acid;
about 100 μg/g to about 200 μg/g compound of unknown identity with the same molecular mass of ferulic acid;
about 100 μg/g to about 250 μg/g p-coumaric acid;
about 10 μg/g to about 50 μg/g hispidulin;
about 30 μg/g to about 60 μg/g rutin;
about 10 mg/g to about 20 mg/g procyanidin;
about 15 mg/g to about 30 mg/g epicatechin and catechin;
about 5 μg/g to about 15 μg/g naringenin; and about 0 μg/g to about 10 μg/g quercetin.
wherein the extract is stable for about 16 weeks at a temperature of from about 20 to about 30° C. as measured by HPLC.

In some embodiments, provided herein is a Euterpe oleracea extract prepared according to methods described herein, the extract comprising:
about 0.5 μg/g to about 2 μg/g vanillic acid;
about 2 μg/g to about 6 μg/g compound of unknown identity, with the same molecular mass of vanillic acid;
about 5 μg/g to about 50 μg/g ferulic acid;
about 100 μg/g to about 40 μg/g compound of unknown identity with the same molecular mass of ferulic acid;
about 0 μg/g to about 1 μg/g p-coumaric acid;
about 2 μg/g to about 20 μg/g hispidulin;
about 10 μg/g to about 70 μg/g rutin;
about 0 mg/g to about 20 mg/g procyanidin;
about 100 μg/g to about 1,000 μg/g epicatechin and catechin;
about 5 μg/g to about 15 μg/g naringenin; and about 0 μg/g to about 10 μg/g quercetin.
wherein the extract is stable for about 16 weeks at a temperature of from about 20 to about 30° C. as measured by HPLC.

Compositions of the present invention comprising the Euterpe oleracea extracts can include additional ingredients such as cosmetic ingredients and pharmaceutical active ingredients.

Cosmetic Ingredients include but are not limited to UV absorption agents, moisturizing agents, antioxidants, structuring agents, emulsifiers, silicone-containing compounds, essential oils, thickening agents and preservatives.

Pharmaceutical active ingredients include but are not limited to anti-acne agents, anti-inflammatory agents, and steroids.

Also contemplated are kits that include any one of the compositions disclosed throughout the specification and claims. In certain embodiments, the composition is comprised in a container. The container can be a bottle, dispenser, or package. The container can dispense a pre-determined amount of the composition. In certain aspects, the compositions are dispensed in a spray, dollop, or liquid. The container can include indicia on its surface. The indicia can be a word, an abbreviation, a picture, or a symbol.

Also contemplated is a product comprising a composition of the present invention. In non-limiting aspects, the product can be a cosmetic product. The cosmetic product can be those described in other sections of this specification or those known to a person of skill in the art. Non-limiting examples of products include a moisturizer, a cream, a lotion, a skin softener, a foundation, a night cream, a lipstick, a cleanser, a toner, a sunscreen, a mask, or an anti-aging product.

In certain embodiments, the compositions are formulated into topical skin care compositions. The compositions can be cosmetic compositions. In other aspects, the compositions can be included in a cosmetic vehicle. Non-limiting examples of cosmetic vehicles are disclosed in other sections of this specification and are known to those of skill in the art. Examples of cosmetic vehicles include emulsions (e.g., oil-in-water and water-in-oil emulsions), creams, lotions, solutions (e.g., aqueous or hydro-alcoholic solutions), anhydrous bases (e.g., lipstick or a powder), gels, and ointments. In other non-limiting embodiments, the compositions of the present invention can be included in anti-aging, cleansing, or moisturizing products. The compositions can also be formulated for topical skin application at least 1, 2, 3, 4, 5, 6, 7, or more times a day during use. In other aspects of the present invention, compositions can be storage stable or color stable, or both. It is also contemplated that the viscosity of the composition can be selected to achieve a desired.

The compositions in non-limiting aspects can have a pH of about 6 to about 9. In other aspects, the pH can be 1, 2, 3, 4, 5, 6, 7, 8, 9, 10, 11, 12, 13, or 14. Compositions of the present invention can have UVA and UVB absorption properties. The compositions can have a sun protection factor (SPF) of 2, 3, 4, 5, 6, 7, 8, 9, 10, 11, 12, 13, 14, 15, 20, 25, 30, 35, 40, 45, 50, 55, 60, or more, or any integer or derivative therein. The compositions can be sunscreen lotions, sprays, or creams.

In another aspect, provided herein is a method of treating the skin of a subject, the method comprising contacting the skin with an effective amount of a Euterpe oleracea extract produced according to methods described herein.

In some embodiments, treating the skin of a subject comprises inhibiting metalloproteinase-1 activity in the skin of a subject, the method comprising contacting the skin with an effective amount of a Euterpe oleracea extract produced according to methods described herein.

In some embodiments, treating the skin of a subject comprises reducing the concentration of reactive oxygen species in the skin of a subject in need thereof, the method comprising contacting the skin with an effective amount of an Euterpe oleracea extract produced according to methods described herein.

In some embodiments, treating the skin of a subject comprises reducing cellular and functional damage in skin of a subject exposed to ultraviolet B (UVB) radiation, the method comprising contacting the skin with an effective amount of an Euterpe oleracea extract produced according to methods described herein.

In some embodiments, treating the skin of a subject comprises reducing wrinkles, fine lines and other dermatological signs of aging in the skin of a subject in need thereof, the method comprising topically applying to the skin an effective amount of an Euterpe oleracea extract produced according to methods described herein.

In some embodiments, treating the skin of a subject comprises preventing photocarcinogenesis in the skin of a subject in need thereof, the method comprising topically applying to the skin an effective amount of an Euterpe oleracea extract produced according to methods described herein.

In some embodiments, treating the skin of a subject comprises treating the skin of a subject comprises preventing UV related skin damage in the skin of a subject in need thereof, the method comprising topically applying to the skin an effective amount of an Euterpe oleracea extract produced according to methods described herein.

In some embodiments, treating the skin of a subject comprises relieving itching or discomfort in the skin of a subject in need thereof, the method comprising topically applying to the skin an effective amount of an Euterpe oleracea extract produced according to methods described herein.

In some embodiments, treating the skin of a subject comprises treating the skin of a subject comprises providing a smoother appearance in the skin of a subject in need thereof, the method comprising topically applying to the skin an effective amount of an Euterpe oleracea extract produced according to methods described herein.

In some embodiments, contacting the skin comprises contacting the dermis and the epidermis, and topically applying to the skin comprises applying to the dermis and the epidermis.

### Methods of Use

The extracts and compositions made according to the methods disclosed herein have a number of therapeutic and cosmetic applications.

The extracts and compositions can be used in various forms of skin care, including as an antioxidant, photoprotector, for soothing skin discomfort, and to reduce or prevent the effects of aging on the skin.

In an aspect, provided herein is a method of use as a cosmetic to improve the appearance of the skin if a subject, the method comprising topically applying (contacting the skin) with an effective amount of an Euterpe oleracea extract produced according to methods described herein to a subject

In an aspect, provided herein is a method of treating or preventing a skin condition comprising topically applying (contacting the skin) with an effective amount of an Euterpe oleracea extract produced according to methods described herein to a subject having a skin condition or at risk of having a skin condition, wherein topical application of the composition to the skin condition treats the skin condition or prevents the skin condition from forming. In particular embodiments, the skin condition is a fine line or wrinkle, dry or flaky skin, erythema, sensitive skin, or inflamed skin. In particular aspects, erythema, sensitive skin, or inflamed skin is caused by skin sunburn, electrical treatments of skin, skin burns, contact allergies, systemic allergies, skin toxicity, exercise, insect stings, bacterial infection, viral infection, fungal infection, protozoa infection, massage, or windburn. In other aspects, the following additional skin conditions can be treated or prevented in accordance with the methods and compositions disclosed throughout the specification and claims: pruritus, spider veins, lentigo, age spots, senile purpura, keratosis, melasma, blotches, nodules, sun damaged skin, dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, acne, impetigo, erysipelas, erythrasma, eczema, and other inflammatory skin conditions. In certain non-limiting aspects, the skin condition can be caused by exposure to UV light, age, irradiation, chronic san exposure, environmental pollutants, air pollution, wind, cold, heat, chemicals, disease pathologies, smoking, or lack of nutrition. The skin can be facial skin or non-facial skin (e.g., arms, legs, hands, chest, back, feet, etc.). The method can further comprise identifying a person in need of skin treatment.

The method can also include topically applying an effective amount of an Euterpe oleracea extract produced according to methods described herein to: increase the stratum corneum turnover rate of the skin; increase collagen synthesis in fibroblasts; increase cellular anti-oxidant defense mechanisms (e.g., exogenous additions of anti-oxidants can bolster, replenish, or prevent the loss of cellular antioxidants such as catalase and glutathione in skin cells (e.g., keratinocytes, melanocytes, langerhans cells, etc.) which will reduce or prevent oxidative damage to the skin, cellular, proteins, and lipids); inhibit melanin production in melanocytes; reduce or prevent oxidative damage to skin (including reducing the amount lipid peroxides and/or protein oxidation in the skin).

In another embodiment, there is disclosed a method of reducing pain associated with erythema, sensitive skin, or inflamed skin, comprising topically applying an effective amount of an Euterpe oleracea extract produced according to methods described herein to erythemic, sensitive, or inflamed skin, wherein topical application of the composition to erythemic, sensitive, or inflamed skin reduces the pain associated with erythema, sensitive skin, or inflamed skin.

In another aspect, there is disclosed a method of increasing collagen production in a skin cell comprising topically applying an effective amount of an Euterpe oleracea extract produced according to methods described herein to a skin cell in need of collagen production, wherein the topical application of the composition to the skin cell increases collagen production in the skin cell. Non-limiting examples of such cells include human epidermal keratinocyte, human fibroblast dermal cell, human melanocytes, three-dimensional human cell-derived in vitro tissue equivalents comprising human keratinocytes, human fibroblasts, or human melanocytes, or any combination thereof (e.g., combination of human keratinocytes and human fibroblasts or a combination of human keratinocytes and human melanocytes).

In some embodiments, treating the skin of a subject comprises inhibiting metalloproteinase-1 activity in the skin of a subject, the method comprising contacting the skin with an effective amount of an Euterpe oleracea extract produced according to methods described herein.

In some embodiments, treating the skin of a subject comprises reducing the concentration of reactive oxygen species in the skin of a subject in need thereof, the method comprising contacting the skin with an effective amount of an Euterpe oleracea extract produced according to methods described herein.

In some embodiments, treating the skin of a subject comprises reducing cellular and functional damage in skin of a subject exposed to ultraviolet B (UVB) radiation, the method comprising contacting the skin with an effective amount of an Euterpe oleracea extract produced according to methods described herein.

In some embodiments, treating the skin of a subject comprises reducing wrinkles, fine lines and other dermatological signs of aging in the skin of a subject in need thereof, the method comprising topically applying to the skin an effective amount of an Euterpe oleracea extract produced according to methods described herein.

In some embodiments, treating the skin of a subject comprises preventing photocarcinogenesis in the skin of a subject in need thereof, the method comprising topically applying to the skin an effective amount of an Euterpe oleracea extract produced according to methods described herein.

In some embodiments, treating the skin of a subject comprises treating the skin of a subject comprises preventing UV related skin damage in the skin of a subject in need thereof, the method comprising topically applying to the skin an effective amount of an Euterpe oleracea extract produced according to methods described herein.

In some embodiments, treating the skin of a subject comprises relieving itching or discomfort in the skin of a subject in need thereof, the method comprising topically applying to the skin an effective amount of an Euterpe oleracea extract produced according to methods described herein.

In some embodiments, treating the skin of a subject comprises treating the skin of a subject comprises providing a smoother appearance in the skin of a subject in need thereof, the method comprising topically applying to the skin an effective amount of an Euterpe oleracea extract produced according to methods described herein.

In some embodiments, contacting the skin comprises contacting the dermis and the epidermis, and topically applying to the skin comprises applying to the dermis and the epidermis.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range.

The following terms are intended to have the meanings presented therewith below and are useful in understanding the description and intended scope of the present invention.

These and other exemplary substituents are described in more detail in the Detailed Description, and Claims. The invention is not intended to be limited in any manner by the above exemplary listing of substituents.

### Other Definitions

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) In certain embodiments, the subject is a human. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). Cosmetic conditions are also included in the conditions which can undergo “treatment,” which may include improvement of skin features rather than treatment of a disorder or defect (“cosmetic treatment”).

“Topical application” means to apply or spread a composition onto the surface of keratinous tissue. “Topical skin composition” includes compositions suitable for topical application on keratinous tissue. Such compositions are typically dermatologically-acceptable in that they do not have undue toxicity, incompatibility, instability, allergic response, and the like, when applied to skin. Topical skin care compositions of the present invention can have a selected viscosity to avoid significant dripping or pooling after application to skin.

In general, the “effective amount” of a compound refers to an amount sufficient to elicit the desired biological response, e.g., to treat skin disorder, is sufficient to induce anesthesia or sedation. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, “Euterpe oleracea material” refers to fruit of the Euterpe oleracea plant, including seeds.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Abbreviations

ACN: Acetonitrile; DCFH-DA: 2′,7′-dichlorofluorescein diacetate; DMSO: Dimethyl sulfoxide; DPPH: α-diphenyl-β-picrylhydrazyl; EL: Extractor liquid; EROs: Essential for reactive oxygen species (a protein); HPLC: High performance liquid chromatography; LC: Liquid chromatography; MI: Mutagenicity index; MSMS: Mass spectrometry-Mass spectrometry (tandem mass spectrometry); MTT: 3-(4,5-dimethylthiazol-2-yl)-2,5-diphenyltetrazolium bromide; PBS: Phosphate buffered saline; ROS: Reactive oxygen species; SDS: sodium dodecylsulfate; ST-035: an extract of Euterpe Oleracea, same as TI-35; TI-35: an extract of Euterpe Oleracea, same as ST-035; UVB: ultraviolet radiation type B; VM: Vegetable matter.

### Example 1. Process for Production of Euterpe Oleracea Hot Extract

Approximately 1.0 kilo of extract was prepared. The procedures involved in the extraction process were: 1) cleaning of the material; 2) dry milling and extraction; or wet milling/maceration, 3) filtration, 4) evaporation, 5) lyophilization or spray drying; and 6) storage.

In the “Industrial Process”, step 2 is dry milling and extraction in a reactor, and step 5 is spray drying.

### Cleaning Stage

55 kilos of Euterpe oleracea fruit including seeds were acquired. After cleaning, the material was washed in running water to eliminate impurities.

The next step was to remove the excess water, letting it dry at room temperature. Later, the material was packed in clean plastic bags and stored in the freezer at a temperature of −20° C. until the time of its use.

### Dry Milling and Extraction Stage

The milling step starts with the washed Euterpe oleracea material being processed in a hammer mill with an 8 mm mesh sieve. The material which was not retained by the sieve was then passed into the extraction step.

In this higher temperature extraction, the hydroalcoholic extract is agitated in a reactor at temperatures above 30° C. for less than 24 hours. For example, the higher temperature maceration can be performed in a cylindrical batch reactor with paddle agitation, at 40 Hz agitation speed, at a temperature of 40+/−20° C., with extractor liquid of 1:1 ethanol/water, and a ratio of particulate material to extractor liquid of about 1:2 to 1:10 and total extraction time of about 1-5 hours.

### Wet Milling/Maceration Stage

The initial extractions were performed with portions of 200.0 grams (approximately) of the cleaned material obtained in the cleaning stage in the proportion of 1 part cleaned material (vegetable matter; VM) to 4 parts extractor liquid (EL), and the EL used for extraction was a mixture of ethanol absolute and deionized water in the proportion of 1:1 (v/v).

The vegetable material was weighed in a glass beaker and washed thoroughly with water to eliminate any impurity; a volume of deionized water was added to the beaker equivalent to twice the weighed mass (for example, for 200.0 grams of VM, 400.0 mL of water was added). The content of the beaker was transferred to the stainless steel container and the material was crushed for approximately 1 minute. The mass of crushed material and water was transferred again to the beaker and the content was boiled for 10 minutes. The beaker with the aqueous extract was left to cool in an ice bath until it reached a temperature of approximately 30° C.

Finally, the content of the beaker was transferred to the maceration container (stainless steel container sealed with Parafilm) and ethanol absolute was added using the same measure that had been used for water, resulting in the proportion of 1 part of vegetable matter (VM) to 4 parts of extractor liquid (EL).

The maceration time of the hydroalcoholic extract 50% (v/v) was from 7 to 10 days in a refrigerator, at a temperature of 4° C. approximately.

### Filtration Stage

After the maceration period in the wet milling/extraction stage, the extract was filtered by gravity in “voil” type fabric to remove the “thick” vegetable material (in bigger particles) and then it was vacuum filtered in 80.0 g/m² filter paper, 3 microns porosity, to remove the smaller size particulate material, produced by the grinding of the seeds. The hydroalcoholic extract was kept in a refrigerator, at 4° C., in amber glass or wrapped in aluminum foil (closed), until the moment of evaporation of the solvent in the rotary evaporator.

After the dry milling and extraction stage, the extract was filtered through a bag filter having a mesh opening of 90 to 187μ, preferably 154μ.

### Rotary Evaporation Stage

After the filtration process, the hydroalcoholic extract 50% (v/v) was subjected a rotary evaporation process to eliminate the organic solvent. The volume of solution placed in the evaporation flask was always less than half of the total volume of the flask (around 800.0 mL each time). For the ethanol evaporation process, two rotary evaporators were used: one from the Solab brand (model SL-126) and another from the Buchi brand (model R-100); both with heating bath, chiller for cooling and vacuum pump. In both kinds of evaporators, the temperatures of the heating bath and chiller were adjusted to 60° C. and 10° C., respectively. For example, the concentration can be performed under vacuum of 60-80 KPa, temperature of 40-70° C., and constant agitation at 30 Hz. This produces concentrated hydroalcoholic extract.

### Pasteurization

Optionally, the extract can be pasteurized before the lyophilization or spray-drying step. This pasteurization can be performed at a variety of temperatures and conditions, preferably below the boiling point of the extract.

### Lyophilization Stage

After the evaporation of the ethanol from the extract, the resulting solution was fractionated in borosilicate glass containers, wrapped in aluminum foil and closed with Parafilm. The amount of extract placed in each container corresponded to a maximum of half of its volume (for example, in a 250.0 mL beaker it was placed around 125.0 mL of extract). After the fractioning, the extracts were frozen at −20° C. for at least 24 hours before the lyophilization process. This produces a frozen hydroalcoholic extract.

The transformation of 50% (v/v) frozen hydroalcoholic extract into dry extract can be accomplished by the lyophilization process, during which the water passes from the solid state (ice) to the gaseous state. This physical state change only occurs with water at very low temperatures (−55° C.) and under vacuum. A lyophilizer from Terroni brand (model LS3000) was used, with capacity for sixteen flasks (or 3.0 kg of ice counting with the containers). The procedure consisted of freezing the extracts for at least 24 hours, perforating the Parafilms that were used to cover the bottles with a needle and placing the containers in the equipment, with the temperature previously stabilized.

The lyophilization process took between 7 and 10 days until the extract was totally dry, depending on the volume of extract in each container.

### Spray Drying Stage

As an alternative to lyophilization, the transformation of concentrated hydroalcoholic extract into dry extract can be accomplished by the spray drying process. For example, the spray drying may be accomplished by optionally adding spray drying excipients, then spray drying with an initial temperature of 190+/−10° C. and final temperature of 95+/−10° C. and a spray dryer disk velocity of 300+/−90 Hz. For example, the spray drying excipients added to the concentrated purified extract can be 40-60% w/v, including SiO₂ and maltodextrin based on the volume of concentrated purified extract prior to spray drying.

In some examples, the Euterpe oleracea extract produced by either lyophilization or spray drying has a maximum water content of 6%.

### Dry Extract Storage

At the end of the lyophilization process, the resulting amount of dry extract in each container was weighed into a previously weighed flask. The extracts were homogenized and aliquoted in bottles wrapped in aluminum foil and stored in a freezer at −20° C. until the moment of use. The extraction method used guaranteed a yield of 4.5%, approximately.

### Chemical Extract Characterization

### High Performance Liquid Chromatography Analysis Coupled to Mass Spectrometry

The samples of the extract were submitted to screening analysis, and the substances that were identified had their confirmation carried out through the analytical pattern addition test.

### Equipment Used for the Characterization of the Compounds Present in the Extract

The samples were analyzed using UPLC-MSMS type equipment (Ultra Performance Liquid Chromatography; MSMS, Mass Spectrometry in tandem) whose system is composed of a Xevo TQS mass spectrometer with a Waters (United Kingdom) triquadrupole mass analyzer. The mass spectrometer is coupled to a high efficiency liquid chromatograph (Acquity H-Class) equipped with a degasser, quaternary pump system, chromatographic column oven, temperature-controlled sampler and automatic injector, also branded Waters. The data acquisition and treatment were performed with the MassLynx software, version 4.1

The mass spectrometer was calibrated in both positive and negative ion modes with a standard phosphoric acid calibration solution. Nitrogen was used as nebulizer gas (flow rate of 150 L/hour) and drying gas (flow rate of 1,000 L/hour). Other MS parameters: ionization source, electrospray; capillary voltage, 4,000 V; source temperature, 500° C.; cone voltage, 40 V. The parameters of the collision cell were: collision gas flow (argon), 0.15 mL/min; collision energy, 28 eV.

### Analytical Method for the Determination of the Extract Constituents

For the quantification of the compounds identified in the extract, two analytical methods had to be developed, as the first methodology proposed was not adequate for the compounds catechin, epicatechin and procyanidin, a fact observed during the validation process of the method.

The Phenolic-MeOH method was used for the analysis of the ferulic acid, rutin, quercetin, vanillic acid, naringenin and hispidulin compounds. The catechin, epicatechin and procyanidin B2 compounds were analyzed in the method called Phenolic-ACN. In both methods, a reversed-phase C18 chromatographic column (Kinetex 50 mm×2.1 mm, particle size 2.6 μm, PheNamenex mark) was used.

The constituent solvents of the mobile phase of the Phenolic-MeOH method were: formic acid 0.1% in deionized water (solvent A) and methanol (solvent B), respectively. In the Phenolic-ACN method, solvent A was also formic acid 0.1% in deionized water and solvent B was formic acid 0.1% in acetonitrile.

In both methods, the linear gradient elution mode provided the best separation of the analytes from other matrix interferents; the flow rate of the mobile phase was fixed at 400.00 μL min−1. In all test runs, the injected volume was 1.00 μL. The oven temperature was adjusted to 30° C. for all analytical runs, and the temperature of the sampler was kept at 10° C. whenever samples, quality controls or calibrators remained inside the equipment. Tables 1 and 2 show the gradient programming used in the Phenolic-MeOH and Phenolic-ACN methods, respectively.

TABLE 1
Mobile Phase for Phenolic-MeOH method

| Time (min) | % MeOH |
|---|---|
| 0.00 | 5 |
| 0.17 | 5 |
| 1.17 | 60 |
| 2.17 | 70 |
| 3.17 | 100 |
| 5.33 | 100 |
| 5.50 | 5 |
| 7.00 | 5 |

TABLE 2
Mobile Phase for Phenolic-ACN Method

| Time (min) | % Acetonitrile |
|---|---|
| 0.00 | 5 |
| 2.00 | 40 |
| 3.00 | 40 |
| 3.10 | 5 |
| 5 | 5 |

### Validation of Analytical Methods

The proposed analytical methods were validated only for the compounds identified in the extract by the standard addition test, which were ferulic acid, rutin, quercetin, vanillic acid, naringenin and hispidulin in the Phenolic-MeOH method. The catechin, epicatechin and procyanidin B2 compounds were validated in the Phenolic-ACN method.

### Linearity/Calibration Curve

For the linearity evaluation of the Phenolic-MeOH method, a methanol calibration curve was constructed in the application range of 25.0 to 475.0 ng mL−1. The calibrators were prepared as follows: first, an individual stock solution was made with each Reference Substance (RS) at the final concentration of 10.0 μg mL−1 from the dilution of the stock solutions 1.0 mg mL−1; then a working solution at the concentration 500.0 ng mL−1 Was made from the dilution of the solution 10.0 μg mL−1 with the six compounds together. After the calibrators preparation procedure the curve with the desired application range was obtained.

### Analysis of Compounds in Hot Extracts

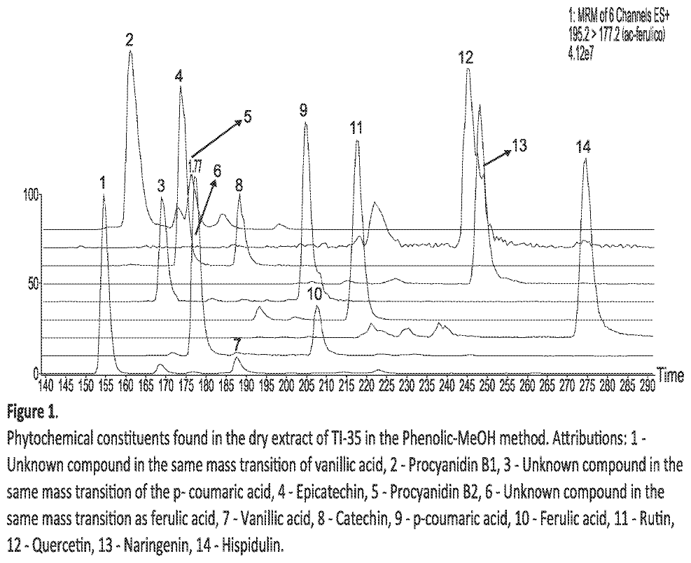

The results of four runs of a hot extract prepared as above, TI-035, are shown in table 3. An example overlay LC trace of compounds in TI-35 is shown in FIG. 1.

TABLE 3
Levels of Some Chemical Compounds in TI-35 Batches

| Compound | Batch 1 | Batch 2 | Batch 3 | Batch 4 |
|---|---|---|---|---|
| Naringenin (μg/g) | 24.63 | 15.07 | 19.33 | 12.91 |
| Quercetin (μg/g) | 6.03 | 3.89 | 4.84 | 1.81 |
| Vanillic Acid (μg/g) | 9.76 | 5.31 | 3.05 | 4.29 |
| Isovanillic Acid (μg/g) | 44.28 | 53.10 | 45.45 | 23.54 |
| Ferulic Acid (μg/g) | 119.02 | 42.69 | 41.6 | 28.05 |
| Isoferulic Acid (μg/g) | 64.73 | 118.72 | 103.50 | 86.46 |
| Hispidulin (μg/g) | 79.25 | 40.92 | 48.61 | 24.52 |
| Rutin (μg/g) | 59.08 | 91.94 | 69.86 | 42.19 |

### Example 2. Biological Analysis of Hot Extracts

In the following assays, the hot extract may be described as TI-35 or ST-035, which terms are used interchangeably.

### Test Substance, Reference Substances and Vehicle Preparation

With the exception of weighing, the handling of Test Substance, Reference Substances and Vehicle was performed under sterile conditions in a biological safety cabinet. The stock solution (SS) of ST-035 Test Substance in a concentration of 25 mg/mL was prepared in DMSO 100%. Subsequently, such solution was placed in the ultrasound device for 10 minutes so as to solubilize the ST-035. Later, concentrations of 0.01, 0.03, 0.1, 0.3, 1, 3, 10, 30 and 100 μg/mL were prepared from the SS with supplemented culture medium or phosphate buffer (PBS), depending on the methodology employed. The Vehicle group or control was prepared precisely as the ST-035, by replacing the ST-035 25 mg/mL with DMSO 100%. The maximum percentage of DMSO found in the ST-035 concentrations was 0.4%, which does not interfere in the tests described below performed with tissue from both lineages (human keratinocytes and fibroblasts). The ascorbic acid reference substance was solubilized in supplemented culture medium. The ferulic acid Reference Substance however, was solubilized in DMSO 100% so as to form a 2 mg/mL stock solution, with subsequent dilutions for the preparation of the 4 μg/mL concentration, which contains 0.2% DMSO.

### Evaluation of α-Diphenyl-β-Picrylhydrazyl (DPPH) Activity

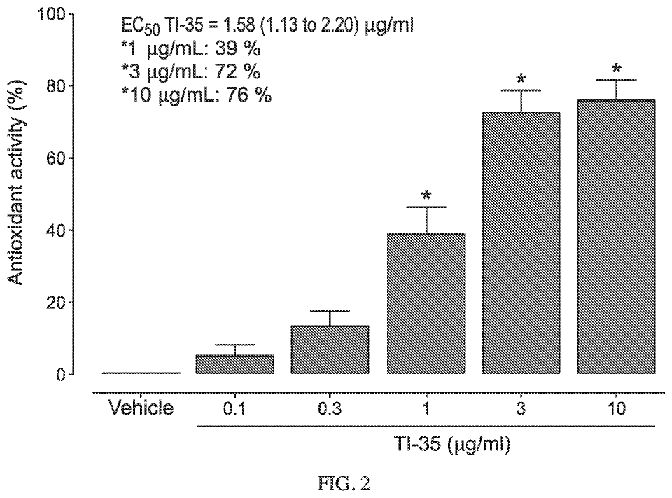

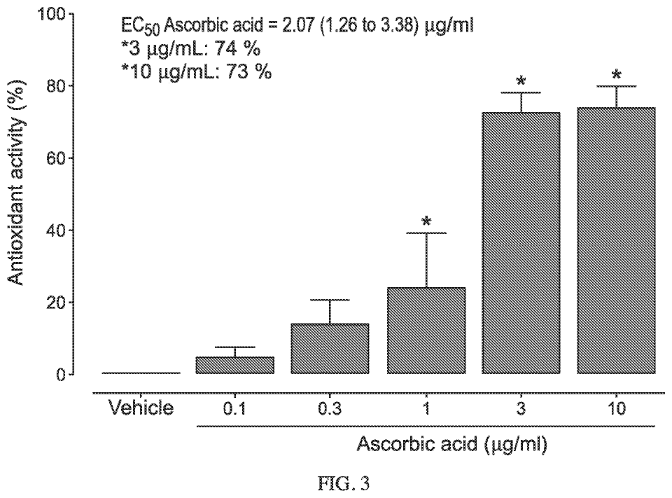

The concentrations of Test Substance and Reference Substance, ascorbic acid (0.1, 0.3, 1, 3 or 10 μg/mL) were prepared according to the description above (Test Substance, Reference Substances and Vehicle Preparation) and 225 μL of each concentration were added to the wells. Then 75 μL of methanol (DPPH vehicle) or 60 μM DPPH solution were added and after 30 to 40 minutes the reading was performed in spectrophotometer at 515 nm (SpectraMax i3x—Molecular Devices). The percentage of DMSO in the Vehicle group was 0.04% (equivalent to the percentage of DMSO at a concentration of 10 μg/mL). As shown in FIGS. 2 and 3, TI-35 has indirect antioxidant activity and is more potent than ascorbic acid.

### Evaluation of Cell Viability Through the MTT Method

For this assay, 7×10³ human keratinocyte cells/well (HEKn) were plated in 96-well culture plates. After approximately 24 hours incubation in a CO₂ incubator at 37±0.1° C. and 5±0.1% CO₂, the culture medium was replaced by a medium containing Vehicle (DMSO 0.12%) or Test Substance at concentrations of 1, 3, 10 or 30 μg/mL and incubated again under the same conditions.

After the incubation times (24, 48 or 96 hours), the cellular viability was analyzed by means of the MTT method as follows:

The cellular viability assay was performed on both lineages (keratinocytes and fibroblasts) through the MTT reduction colorimetric test, in which viable cells reduce the MTT salt, forming a formazan complex inside their mitochondria. For this assay, after the different incubation times with treatments (24, 48 or 96 hours for keratinocytes and 24 or 48 hours for fibroblasts), the culture medium was exchanged for a medium containing MTT (0.5 mg/mL) and the cells were incubated for approximately 4 hours at 37° C.±0.1° C. in a humidified atmosphere containing 5%±0.1% CO2. Subsequently, the MTT solution was removed and 100 μL of dimethylsulfoxide (DMSO; 100%) was added. Absorbance was measured at 570 nm, using the SpectraMax i3x equipment (Molecular Devices). The results were expressed as a percentage of viable cells in relation to the control (Vehicle group, containing 0.12% DMSO).

The concentrations of the Test Substance (1, 3, 10 and 30 μg/mL) were prepared according to the description above (Test Substance, Reference Substances and Vehicle Preparation).

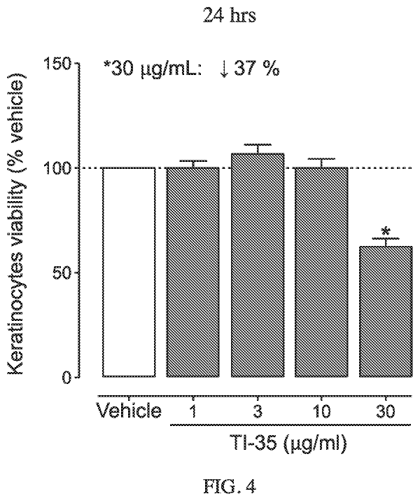

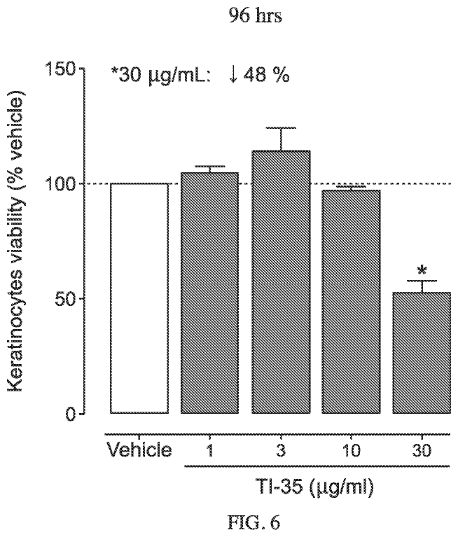

As shown in FIGS. 4-6, the vehicle group (0.12% DMSO) and ST-035 at concentrations of 1, 3 and 10 μg/mL do not alter the keratinocyte viability after 24 hours (A), 48 hours (B) or 96 hours (C) of incubation. However, the 30 μg/mL concentration reduces viability by 37.3±3.7%, 43.6±6.1% and 47.5±5.7% in 24, 48 and 96 hours, respectively. As the concentration of 30 μg/mL was cytotoxic, in subsequent assays such concentration was discarded.

### Evaluation of Cellular Proliferation Using the Sulforhodamine B Method

7×10³ human keratinocyte cells/well (HEKn) were plated in 96-well culture plates. After approximately 24 hours of incubation, a time group for evaluation of time 0 (T0) was fixed with 20% trichloroacetic acid solution and for the rest, the culture medium was replaced by a medium containing Vehicle (DMSO 0.04%) or Test Substance at concentrations of 1, 3 or 10 μg/mL, which were incubated again under the same conditions. After 96 hours, cellular proliferation was analyzed through the Sulforhodamine B method.

The evaluation method for the antiproliferative activity of the compounds was performed according to the methodology described by Vichai, V. and Kirtikara, K., Sulforhodamine B colorimetric assay for cytotoxicity screening, Nat Protoc. 2006; 1(3):1112-6. This method analyzes cell proliferation through the ability of sulforhodamine B to bind to protein components of cells that are fixed by trichloroacetic acid. For this assay, keratinocytes were plated, overnight incubated and a group containing untreated cells was fixed as described below and maintained at room temperature until the end of the trial protocol (Time 0—T0). The remaining cells were then treated with the Vehicle group (0.04% DMSO) or Test Substance at concentrations of 1, 3 or 10 μg/mL for 96 hours. After this incubation period, the treatments were removed and 100 μL of cooled 20% trichloroacetic acid was added to each well to fix the cells. After one hour of fixation at 4° C., the plates were washed and left to dry for a few minutes. Subsequently, 50 μL of Sulforhodamine B (0.1%) was added to each well for 30 minutes. Then, the plates were washed with 1% acetic acid for four times. Finally, 100 μL of Trizma base 10 mM were added for solubilization of Sulforhodamine B. The absorbances of the group which was fixed at the initial time (T0) and of the groups that were incubated for 96 hours were quantified at 540 nm using the SpectraMax i3x equipment (Molecular Devices). The results were expressed as a percentage in relation to the control group (vehicle).

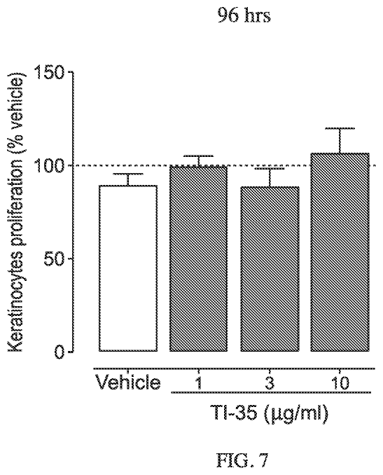

The human keratinocytes were incubated for 96 hours in the presence of the Vehicle group (0.04% DMSO) or Test Substance 1, 3 or 10 μg/mL. The keratinocyte proliferation results are shown in FIG. 7. The values obtained in time 0 were decreased from 96 hours. The bars represent the mean±standard error of the mean of 2 different assays in triplicate. For the statistical analysis, one-way variance analysis (ANOVA) was used, followed by Newman-Keuls test (*p<0.05 in comparison to Vehicle group).

The concentrations of the Test Substance (1, 3 and 10 μg/mL), were prepared according to the description above (Test Substance, Reference Substances and Vehicle Preparation).

As shown in FIG. 7, after 96 hours of incubation with TI-35 concentrations (1, 3 or 10 μg/mL), there was no change in the proliferation of these cells when evaluated by the Sulforhodamine B assay.

### Evaluation of the Antioxidant and Scavenger Activity Through the DCFH-DA Probe

The inhibition of the production of EROs by the Test Substance and Reference Substances (ascorbic acid for keratinocytes; ascorbic acid and ferulic acid for fibroblasts) was evaluated by a fluorimetric method, with the permeable 2′,7′-dichlorofluorescein diacetate (DCFH-DA) probe. The 2′,7′ dichlorofluorescein diacetate (nonfluorescent) is de-esterified intracellularly and becomes a 2′,7′ highly fluorescent dichlorofluorescein after oxidation. Fluorescence detection was performed at 485 nm excitation wavelength and 535 nm emission wavelength in SpectraMax i3x (Molecular Devices) equipment. For the evaluation of the intrinsic antioxidant activity in keratinocytes, the results were expressed as a percentage of the production of EROs compared to the vehicle group (without UVB stimulus—100%). For the rest of the tests using the DCFH-DA probe methodology, the results were expressed as a percentage of the ROS production compared to the vehicle+UVB group (100%), both for keratinocytes and fibroblasts. The concentrations of the Test Substance (10 μg/mL), Ascorbic acid Reference Substance (10 μg/mL) and Ferulic acid Reference Substance (4 μg/mL) were prepared according to the description above (Test Substance, Reference Substances and Vehicle Preparation).

The intrinsic antioxidant activity of Test Substance in human keratinocytes was evaluated as follows. Keratinocytes were incubated with the DCFH-DA probe and after 30 minutes the cells were incubated with the Vehicle group (0.04% DMSO), Test Substance ST-035 (10 μg/mL), ascorbic acid (10 μg/mL) or ferulic acid (4 μg/mL), for 4 or 24 hours. Subsequently, the production of EROs was evaluated through the microplate reader (485/535 nm). Two different assays were performed in triplicate. For the statistical analysis, one-way analysis of variance (ANOVA) was used, followed by the Newman-Keuls test (*p<0.05 in comparison to Vehicle group).

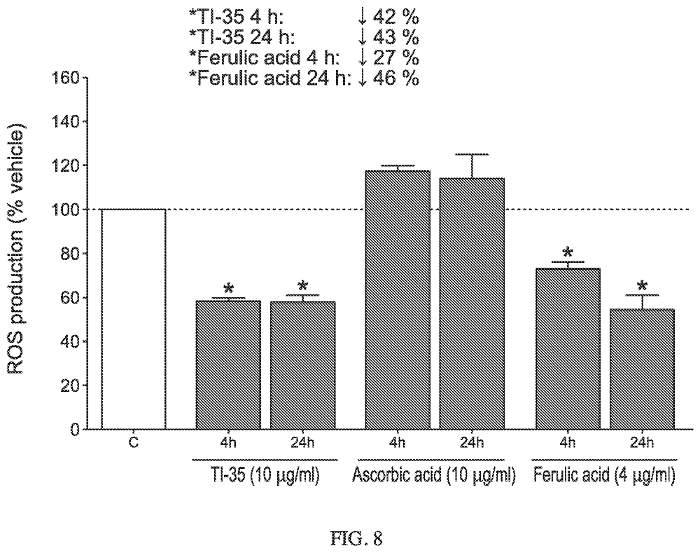

As shown in FIG. 8, the incubation of keratinocytes with ST-035 10 μg/mL reduced the basal production of EROs by 41.7±1.2% and 42.6±3.5%, in the times of 4 and 24 hours, respectively. Ferulic acid (4 μg/mL) also reduced EROs basal production by 26.8±2.4% (4 hours) and 45.8±6.6% (24 hours). However, the ascorbic acid (10 μg/mL) did not change the basal production of EROs, in the two times evaluated.

### Evaluation of Scavenger and Antioxidant Activities of the Test Substance Against UVB Radiation in Keratinocytes

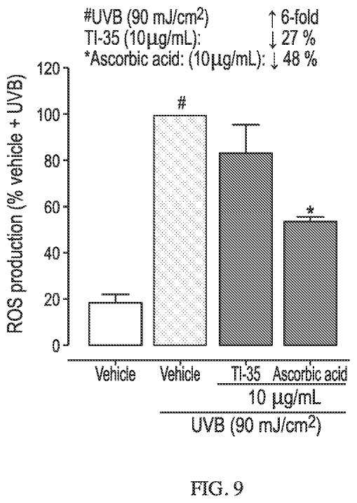

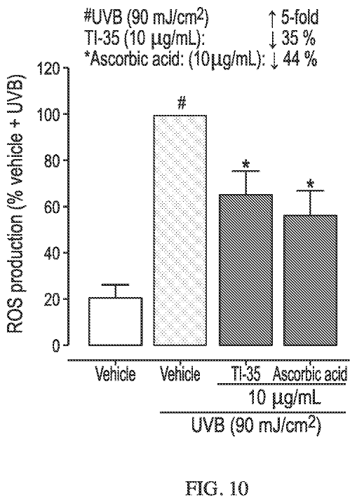

The scavenger potential of the ST-035 Test Substance in human keratinocytes was evaluated. The human keratinocytes were incubated with a DCFH-DA (50 μM) probe for 30 minutes. After this period, the keratinocytes were incubated with the Vehicle group (0.04% DMSO), ST-035 or ascorbic acid (10 μg/mL) and subsequently the cells were irradiated with the UVB 90 mJ/cm² inducing agent. The scavenger activity of the compounds was evaluated by taking the excitation/emission wavelength reading of 485/535 nm, 45 minutes after radiation. The results are shown in FIGS. 9 and 10. The bars represent the mean±standard error of the mean of 3 different assays in triplicate. For the statistical analysis, one-way variance analysis (ANOVA) was used, followed by the Newman-Keuls test (#p<0.05 in comparison to the Vehicle group and *p<0.05 in comparison to Vehicle group+UVB 90 mJ/cm²

FIG. 9 represents the scavenger potential of ST-035 in human keratinocytes with respect to the UVB induced production of EROs. The UVB radiation (90 mJ/cm²) increased the production of the reactive species by 5.4 times. The addition of ST-035 (10 μg/mL) immediately prior to the radiation reduced the production of EROs by 16.4±12.1%. The ascorbic acid reference substance however, reduced by 46.2±2.0%.

The antioxidant potential of the ST-035 Test Substance in human keratinocytes was evaluated as follows. The human keratinocytes were incubated with a DCFH-DA (50 μM) probe for 30 minutes. After this period, the keratinocytes were incubated with the Vehicle group (0.04% DMSO), ST-035 or ascorbic acid for 24 hours and following this time, the cells were irradiated with the UVB 90 mJ/cm² inducing agent. The compounds antioxidant activity was evaluated by taking the excitation/emission wavelength reading of 485/535 nm, 45 minutes after radiation. The results are shown in FIG. 10. The bars represent the mean±standard error of the mean of 3 different assays in triplicate. For the statistical analysis, one-way variance analysis (ANOVA) was used, followed by the Newman-Keuls test (#p<0.05 in comparison to the Vehicle group and *p<0.05 in comparison to Vehicle group+UVB 90 mJ/cm²

The antioxidant capacity (FIG. 10) after pre-Treatment with ST-035 (10 μg/mL) or with ascorbic acid (10 μg/mL) was evaluated after UVB radiation (90 mJ/cm²), which increased the production of the reactive species by 5 times. Incubation for 24 hours before radiation with ST-035 or ascorbic acid reference substance decreased the production of EROs by 35.1±10.4% and 44.3±11.4%, respectively.

### Evaluation of the Metalloproteinase-1 (MMP-1) Enzyme Activity.

The MMP-1 enzyme was evaluated after incubation with the Vehicle (0.04% DMSO), ST-035 (10 μg/mL), Ascorbic acid Reference Substance (10 μg/mL), Ferulic acid Reference Substance (4 μg/mL) or positive control of the test, GM6001, a MMP-1 enzyme activity inhibitor. The bars represent the mean±standard error of the mean of 1 assay performed in triplicate. The percentage of inhibition was calculated in relation to the Vehicle group. For the statistical analysis a one-way analysis of variance (ANOVA) was used, followed by the Newman-Keuls test (*p<0.05 in comparison to Vehicle group).

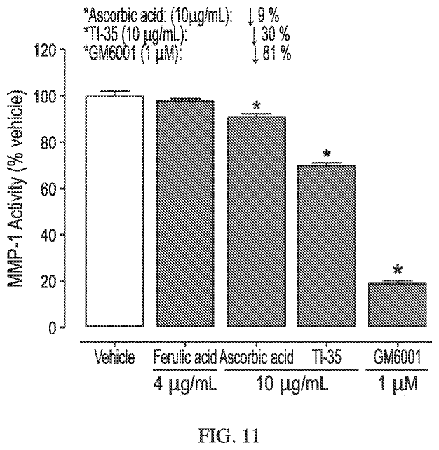

As shown in FIG. 11, TI-35 (10 μg/mL) inhibited the MMP-1 enzyme activity by 30.0±1.0% and the ascorbic acid Reference Substance inhibited only 9.2±1.5%. The ferulic acid did not change in vitro the MMP-1 activity. For the validation of the assay 1 μM of GM6001 (MMP-1 specific inhibitor) was used, which reduced its activity by 81.3±1.5%.

### Evaluation of Fibroblast Cell Viability Through the MTT Method

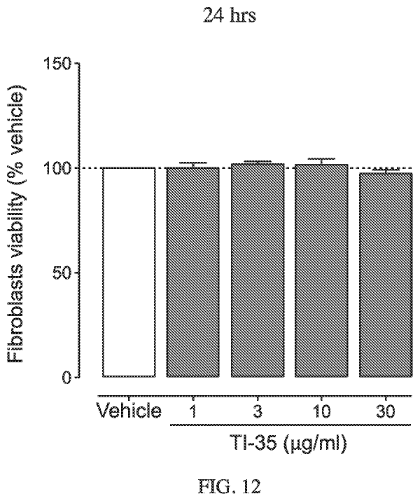

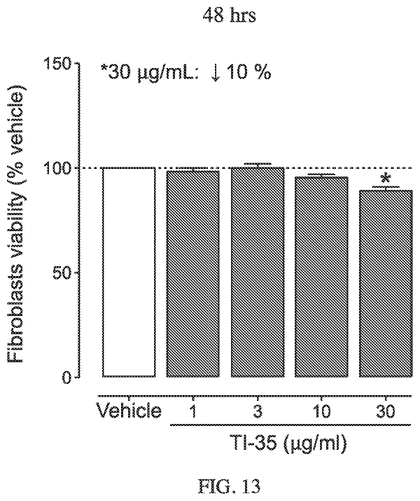

The viability of human fibroblasts was evaluated after incubation for 24 or 48 hours with Test Substance, through MTT assay. The cells were incubated with the Vehicle group (0.12% DMSO) or Test Substance at 1, 3, 10 or 30 μg/mL concentrations for 24 hours (FIG. 12) or 48 hours (FIG. 13) followed by the cell viability assay through the MTT method. The bars represent the mean±standard error of the mean of 4 distinct assays in triplicate. For the statistical analysis, one-way analysis of variance (ANOVA) was used, followed by the Newman-Keuls test (*p<0.05 in comparison to the Vehicle group). The inhibition percentage was calculated in relation to Vehicle group.

The human fibroblast lineage was also evaluated through the MTT assay, as shown in FIGS. 12 and 13. The vehicle group (0.12% DMSO) and ST-035 at concentrations of 1, 3 and 10 μg/mL do not change the viability of fibroblasts after 24 hours (FIG. 12) or 48 hours (FIG. 13) of incubation. However, in the 48 hour-time, the 30 μg/mL concentration reduced viability by 10.5±1.9%; therefore, it is discarded in subsequent assays.

### Evaluation of Scavenger and Antioxidant Activities of the Test Substance Against UVB Radiation in Fibroblasts

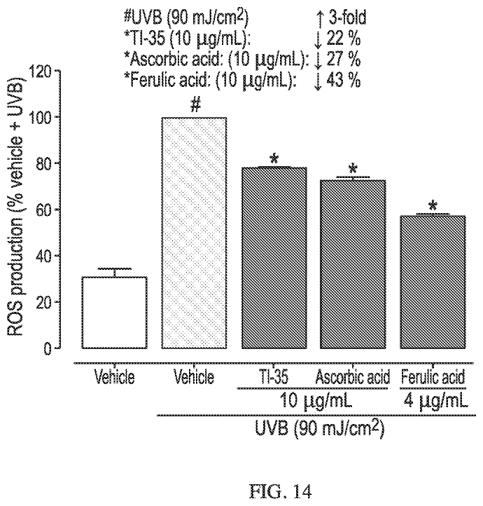

The scavenger potential of the ST-035 Test Substance in human keratinocytes was evaluated. The human keratinocytes were incubated with a DCFH-DA (50 μM) probe for 30 minutes. After this period, the keratinocytes were incubated with the Vehicle group (0.04% DMSO), ST-035 or ascorbic acid (10 μg/mL) and subsequently the cells were irradiated with the UVB 90 mJ/cm2 inducing agent. The scavenger activity of the compounds was evaluated by taking the excitation/emission wavelength reading of 485/535 nm, 45 minutes after radiation. The results are shown in FIG. 14. The bars represent the mean±standard error of the mean of 3 different assays in triplicate. For the statistical analysis, one-way variance analysis (ANOVA) was used, followed by the Newman-Keuls test (#p<0.05 in comparison to the Vehicle group and *p<0.05 in comparison to Vehicle group+UVB 90 mJ/cm2

FIG. 14 represents the scavenger potential of ST-035 in human keratinocytes with respect to the UVB induced production of EROs. The UVB radiation (90 mJ/cm2) increased the production of the reactive species by 5.4 times. The addition of ST-035 (10 μg/mL) immediately prior to the radiation reduced the production of EROs by 16.4±12.1%. The ascorbic acid reference substance however, reduced by 46.2±2.0%.

### Evaluation of the Antioxidant Potential of ST-035 Test Substance in Human Fibroblasts.

The human fibroblasts were incubated with a DCFH-DA probe (50 μM) for 30 minutes. After this period, the fibroblasts were incubated. For statistical analysis, one-way variance analysis (ANOVA) was used, followed by Newman-Keuls test (#p<0.05 compared to Vehicle group and *p<0.05 compared to Vehicle+UVB 90 mJ/cm²).

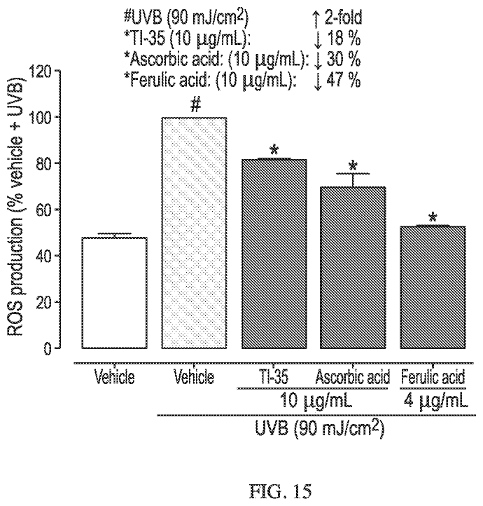

Pre-incubation with ST-035 10 μg/mL reduced the production of reactive oxygen species by 18.4±0.6%. Ascorbic acid, at the same concentration, reduced by 30.5±6.2% and ferulic acid (4 μg/mL) decreased by 47.4±0.5%, as shown in FIG. 15.

### Evaluation of Tyrosinase Enzyme Activity

Tyrosinase enzyme is the limiting step enzyme for melanin formation and responsible for several important steps in its production (Gruber, James V. and Holtz, Robert, Oxid. Med. Cell Longev., 2013; (1):702120. Compounds that act as tyrosinase inhibitors can affect the synthesis of melanin. 10 μL of the concentrations of ST-035 (10, 30, 100, 300 or 1,000 μg/mL), SR-31 (10, 30, 100, 300 or 1,000 μg/mL) or Vehicle were added to the respective wells. Subsequently, 20 μL tyrosinase, (500 U/mL solubilized in a 50 mM sodium phosphate buffer, pH 6.5) were added. To the wells of the “white” group (tyrosinase free group) 20 μL of a sodium phosphate buffer 50 mM (pH 6.5) were added. After that, 170 μL of L-tyrosine substrate 1 mM, solubilized in a potassium phosphate buffer 50 mM (pH 6.5) were added. After the incubation period of 30 to 40 minutes, the plate was read at 490 nm.

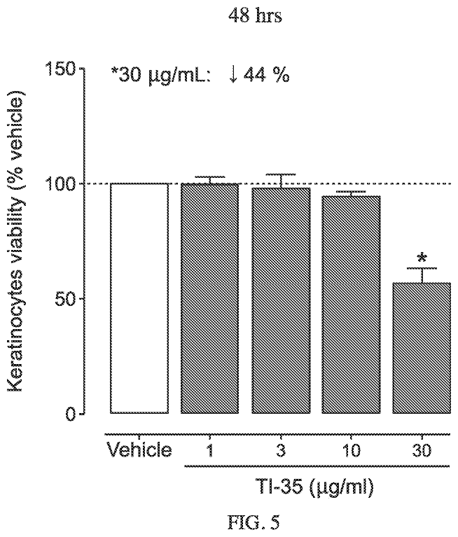

In order to verify the depigmentation potential of ST-035, the first step was to evaluate the activity of tyrosinase, a fundamental enzyme for the synthesis of melanin. A substance that is capable of reducing tyrosinase activity has great potential to be a depigmenting agent. ST-035 concentrations and kojic acid (10, 30, 100, 300 or 1,000 μg/mL) were evaluated and as observed in FIG. 5, no ST-035 concentrations reduced tyrosinase enzyme activity. However, the Reference Substance (kojic acid), showed a reduction of 43 and 84% for the concentrations of 300 and 1,000 μg/mL, respectively. The IC₅₀ value obtained from the Reference Substance was 373.2 μg/mL and confidence limits of 257.5 to 541.0 μg/mL.

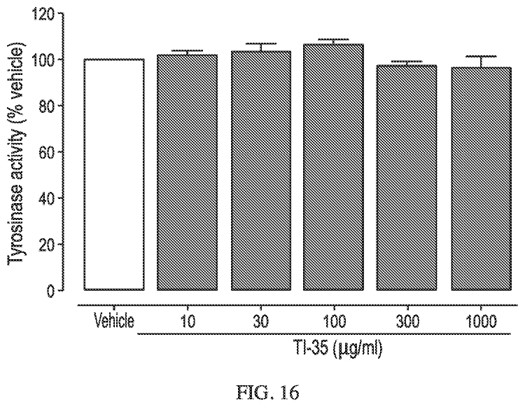

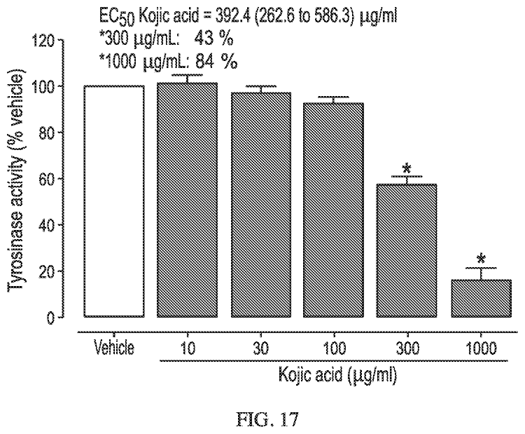

The inhibition of the tyrosinase enzyme was evaluated in the presence of tyrosine substrate and tyrosinase enzyme with different concentrations of Test Substance (FIG. 16) or Reference Substance (FIG. 17). The bars represent the mean±standard error of the mean of 3 different assays in triplicate. For the statistical analysis one-way analysis of variance (ANOVA) was used, followed by the Newman-Keuls test (*p<0.05 in comparison to the Vehicle group). The percentage of inhibition was calculated in relation to the Vehicle group.

### Evaluation of Melanocyte Cell Viability Through the MTT Method

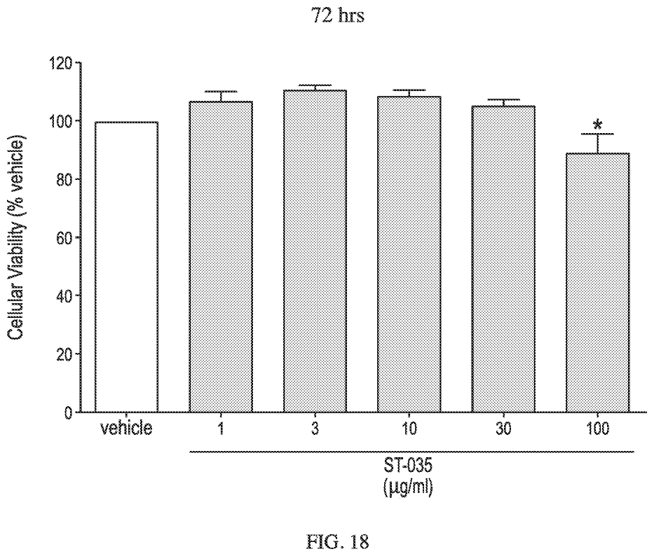

The viability of human melanocytes after incubation for 72 hours with the Test Substance, through the MTT assay was evaluated. The cells were incubated with the Test Substance at concentrations of 1, 3, 10, 30 or 100 μg/mL for 72 hours and then the cell viability test was performed using the MTT method. The results are shown in FIG. 18. The bars represent the mean±standard error of the mean of 3 different assays in triplicate. For the statistical analysis one-way variance analysis (ANOVA) was used, followed by the Newman-Keuls test (*p<0.05 in comparison to the Vehicle group). The percentage of inhibition was calculated in relation to the Vehicle group.

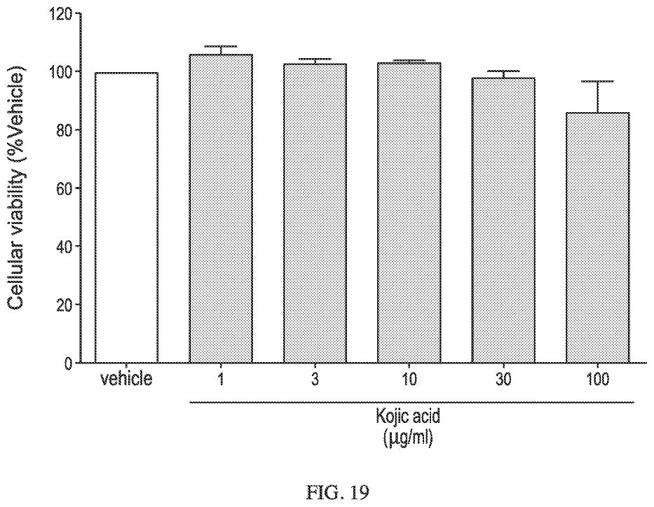

After the 72-hour incubation period, the ST-035 Test Substance (FIG. 18) at a concentration of 100 μg/mL caused a reduction in the viability of human melanocytes, resulting in 88.5±6.7% of viable cells after this period. In the other concentrations evaluated, ST-035 did not change cell viability. The Kojic acid Reference Substance (FIG. 19) was not able to reduce the viability of this lineage.

### Evaluation of Melanin Production

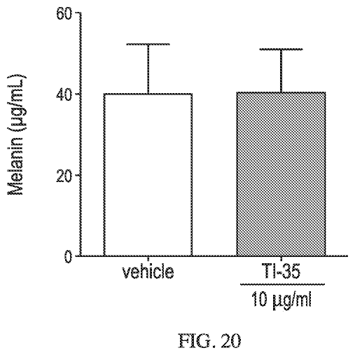

Melanin production in human melanocyte culture was evaluated. Human melanocytes were incubated for 72 hours in the presence of Test Substance 10 μg/mL. The results are shown in FIG. 20. The bars represent the mean±standard error of the mean of 3 distinct assays in triplicate. For statistical analysis the Student t-test was used. The melanin production (in μg/mL) was calculated from a synthetic melanin curve.

Although ST-035 has no effect on tyrosinase activity, the depigmenting potential of a compound can also be evaluated through the direct production of melanin by a mechanism other than enzyme activity. Thus, after incubation of the Test Substance 10 μg/mL for 72 hours with human melanocytes, there was no change in basal melanin production, as shown in FIG. 20.

### Skin Irritation Assay

The possible irritant action of ST-035 on a reconstructed human tissue model, as described in the OECD Guidelines for the Testing of Chemicals, Section 4, TG439 In Vitro Skin Irritation: Reconstructed Human Epidermis Test Method was evaluated. After receiving the tissues, they were readapted in growth medium overnight until the Treatment moment. After the readaptation, Negative Control, positive control (Reference Substance) or Test Substance were added in a 16 μL volume on the epidermis and maintained for 42 minutes (±1 minute). Following, each tissue went through a 25-phosphate buffer washing process to remove the Treatments. Subsequently, the epidermis was maintained for 42 hours (±1 hour) in growth medium in a CO₂ incubator (5±0.1%) at 37±0.1° C. After this period, the tissues were incubated for 3 hours (±15 minutes) with a 1 mg/mL MTT solution and then 2 hours with isopropanol for the formazan complex extraction. Then, 200 μL of each well (in duplicate) were transferred to a 96-well plate for absorbance reading at 570 nm.

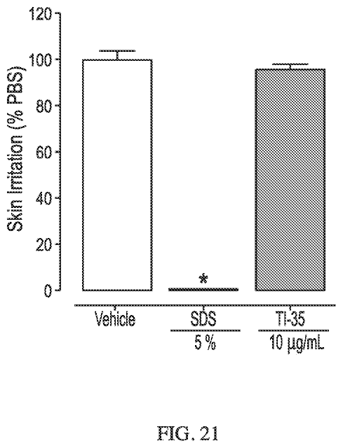

FIG. 21 shows the indirect cellular viability measurement (absorbance at 570 nm) of the incubated tissues with the different Treatments, for 42 minutes. The values refer to three different tissues, for each trial group. Negative Control (PBS) presented a viability mean of 1.760±0.116; positive control (Reference Substance) 0.018±0.001 and the ST-035 Test Substance with viability of 1.692±0.068. In terms of percentage, Negative Control represents 100% tissue viability, positive control (Reference Substance) only 1.02% and Test Substance 96.14%. The model was validated with the results obtained after incubation with the positive control SDS (Reference Substance), which presented viability below 40% (according to the acceptance criteria).

Skin irritation assay in a reconstructed human skin model is an alternative method that follows the OECD TG439 standards and is validated by international centers, with international regulatory acceptance. According to the Globally Harmonized System of Classification and Labeling of Chemicals (GSH), the irritant potential of Test Substances can be distinguished between Category 2 skin irritants and non-classified (do not cause skin irritation).

To be considered irritant (category 2), at the end of the assay the substance must show a reduction of 50% or more in cell viability compared to the Negative Control group. If the substance shows viability above 50% compared to Negative Control, it will be considered non-irritant. According to the GSH criteria, ST-035 is considered non-irritant.

### Mutagenicity Tests of TI-35

The possible mutagenicity potential of test substance ST-035 was evaluated in the strains TA 97a, TA98, TA 100, TA 102 and TA 1535 of S. typhimurium in the absence or presence of metabolic activation with S9, a liver extract that contains active liver enzymes (P450 activity).

In the preliminary test, performed with strain TA 100 in the absence and presence of S9 no mutagenic effect of ST-035 was observed in the selected concentrations (8, 40, 200, 1000 and 5,000 μg/plate). Thus, the same concentrations were used to perform the final test with all strains in the absence or in the presence of S9.

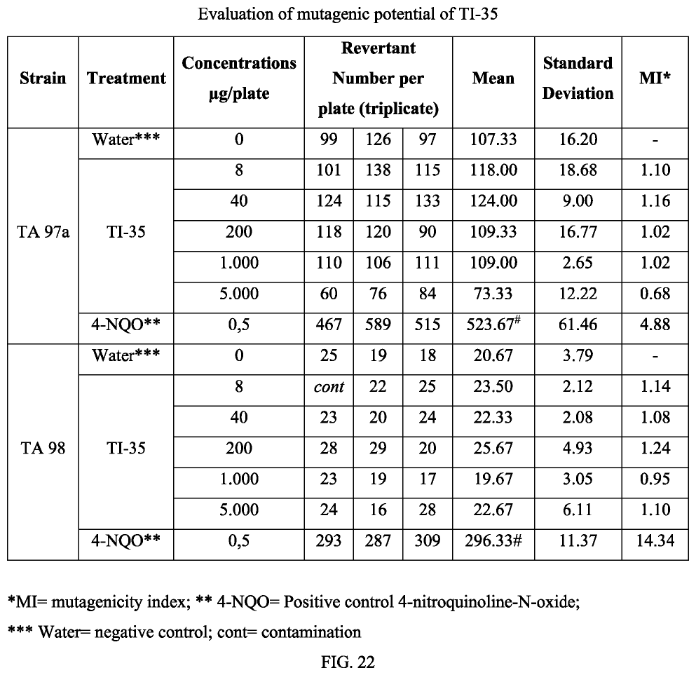

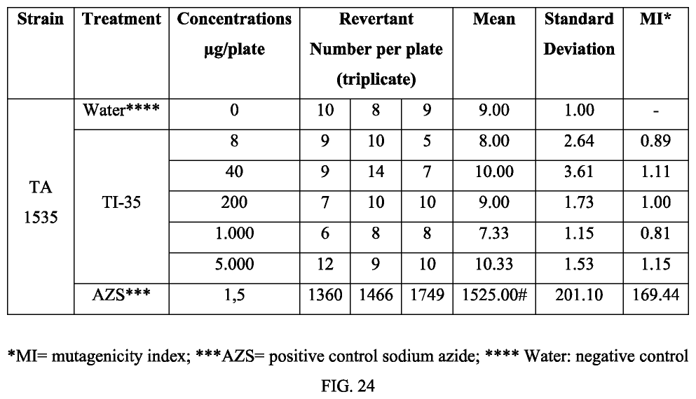

After the Test Substance incubation with the five strains of S. typhimurium, in the absence of S9, no increase in the number of revertant colonies (mutagenicity) was observed, which resulted in mutagenicity index (MI) values between 0.80 and 1.16. At a concentration of 5,000 μg/plate, cytotoxicity was observed in the strains TA 100 and TA 97a, with a mutagenicity index (MI) of 0.52 and 0.68, respectively (FIGS. 22-24).

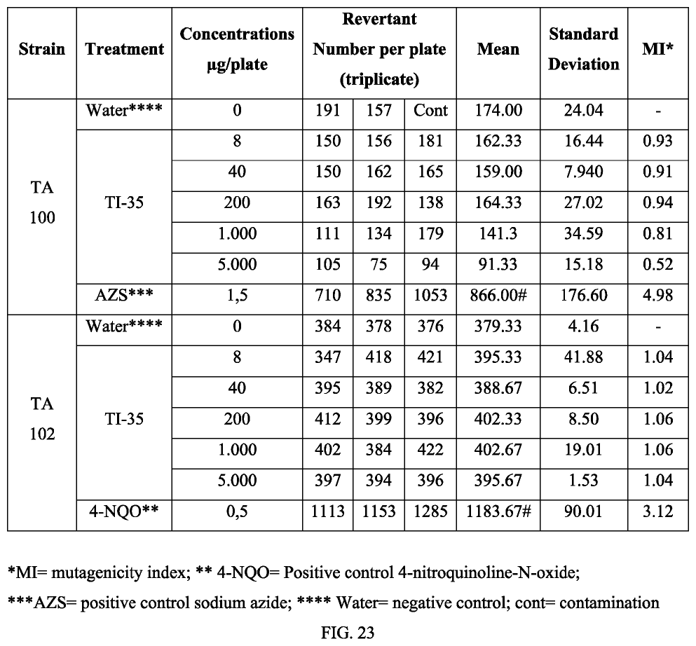

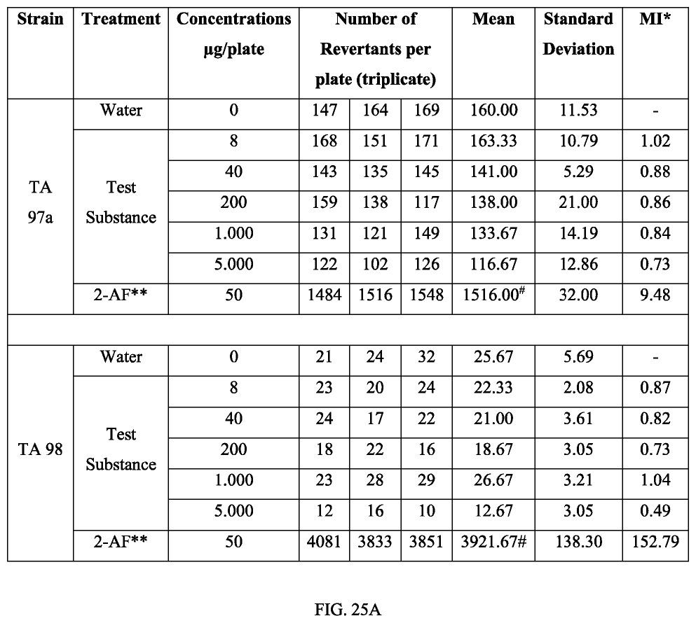

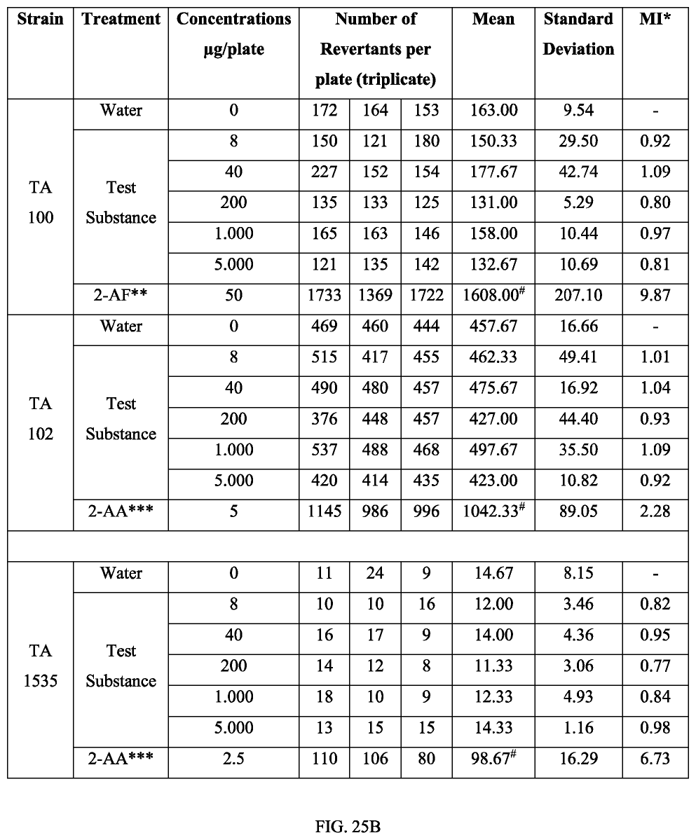

No changes in the number of revertant colonies (mutagenicity) with MI values between 0.73 and 1.09 were observed in the presence of metabolic activation. Cytotoxicity was observed in strain TA 98 only with a concentration of 5,000 μg/plate, with MI of 0.49 (FIGS. 23-25).

As Substances with mutagenicity indexes equal to or greater than 2 for strains TA97a, TA98, TA100 and TA102 or values equal to or greater than 3 for strain TA1535 are considered mutagenic, the results described above meet the criteria for a negative response.

### Example 3. Procedure for Preparation of Cold Extracts and Comparative Hot Extracts

### Maceration Stage

In a previous study with TI-35 a 50% ethanol (v/v) hydroalcoholic extract was standardized with the amounts of: 200 g of vegetable matter and 800 mL of extracting liquid, with the addition of 400 mL of water at room temperature to the vegetable matter before it is crushed in an industrial blender, later heating until the mixture boils and maceration time of seven days in the refrigerator.

In this study, maceration time was reduced from seven to three days; a cereal grinder was used to improve the grinding of the seeds as much as possible instead of using an industrial blender; and different percentages of absolute ethanol mixed with deionized water as an extracting liquid were evaluated, and the percentages varied between 75 and 85% alcohol (v/v) (Extracts B and C). In addition, the boiling step was omitted in the preparation of Extracts B and C.

The procedures used for the production of the extracts were as follows: approximately 200 grams of the vegetable matter ground in a cereal grinder was weighed in a glass beaker and the beaker was added with sufficient volume of extraction liquid (different percentages of ethanol and water) and for the production of the extracts, the mixture was homogenized by mechanical stirring. The maceration time of all hydroalcoholic extracts was three days in the refrigerator, at a temperature of approximately 4° C. The extract made with 75% alcohol was extract B, and the extract made with 85% alcohol was extract C.

A 50% ethanol and 50% water extract, Extract A, was produced in the same way as in the preparation of hot extract TI-35, but with a maceration time of only three days. For this extract, the same procedures used for the other extracts were also used: 200 g of vegetable matter and 800 mL of extraction liquid. This extract was prepared by placing the seeds in 400 mL of water in an industrial blender and crushing for 1 minute. Then the mixture of water and seeds was crushed and placed in a glass beaker and heated until boiling. After cooling the container, 400 ml of ethanol was added to the mixture of water and seeds and the contents were homogenized and kept in the refrigerator at a temperature of 4° C. for three days.

A comparative extraction test was also carried out with the percentage of 50% ethanol as extraction liquid. This extract is similar to that described in EP 2 051 721 B1; however, to the amount of 200 grams of vegetable matter 400 ml of boiling water was added and this mixture was left to stand for 5 minutes. Then the contents of the beaker were transferred to the stainless-steel container of an industrial blender and the material was crushed for approximately 3 minutes; the mass of crushed material and water was transferred back to the beaker and the contents were again boiled for another 10 minutes. Subsequently, the beaker with the aqueous extract was allowed to cool and, only then, absolute ethanol was added using the same measure that had been used for water, resulting in the ratio of 1 part of vegetable matter to 4 parts of liquid extractor. The maceration time of this 50% (v/v) hydroalcoholic extract, Extract D, was kept in a refrigerator at 4° C. for seven days.

For a better understanding, a summary with the extraction tests performed is described below:
Extract A=hot extract with 50% ethanol, macerated for 3 days.
Extract B=cold extract with 75% ethanol, macerated for 3 days.
Extract C=cold extract with 85% ethanol, macerated for 3 days.
Extract D=extract produced exactly like EP 2 051 721 B1: hot extract, with 50% ethanol, macerated for 7 days.

### Filtration and Rotary Evaporation Stage

After the maceration period mentioned above, the extracts obtained were filtered and kept in the refrigerator in flasks wrapped in aluminum foil and closed until the solvent evaporated through the rotary evaporator. Table 4 shows the volumes obtained with each extract after filtration.

TABLE 4
Result of the filtration of the new hydroalcoholic
extracts from Test Item 035 and other extracts.

| | | Final volume after |
|---|---|---|
| Extract | Identification | filtration (mL) |
| A | 50% EtOH | 330 |
| B | 75% EtOH | 590 |
| C | 85% EtOH | 620 |
| D | Comparative Extract D50% EtOH | 620 |

Evaporation was done in a single step with each extract, adding the total volume obtained in the filtration to the evaporation flask. The temperatures of the heating bath and the chiller were adjusted to 60° C. and 10° C., respectively. Under these conditions, the time for the volume of alcohol to be reduced was about two hours. After that period, the resulting extract was considered to be aqueous.

The extracts produced with maceration and lyophilization were analyzed and the results are shown in table 5.

TABLE 5
Components in Extracts A-D

| | Extract | Extract | Extract | Extract |
|---|---|---|---|---|
| | A | B | C | D |
| | (μg/g | (μg/g | (μg/g | (μg/g |
| Compound | extract) | extract) | extract) | extract) |
| Ferulic | 4.7 | 3.4 | 3.0 | 2.9 |
| Acid | | | | |
| Unknown | 27.1 | 17.7 | 7.6 | 17.1 |
| Ferulic acid | | | | |
| analog | | | | |
| Rutin | 26.5 | 51.1 | 38.9 | 17.1 |
| Quercetin | ND | 0.9 | 0.9 | ND |
| Vanillic | 1.6 | 1.8 | 1.2 | 1.1 |
| Acid | | | | |
| Unknown | 26.3 | 44.6 | 29.1 | 8.3 |
| Vanillic | | | | |
| acid analog | | | | |
| Naringenin | ND | 3.1 | 2.6 | ND |
| Hispidulin | 8.5 | 14.6 | 11.2 | 2.8 |
| Catechin | 217.4 | 305.0 | 174.4 | 100.8 |
| Epicatechin | ND | 300.0 | 184.7 | 81.0 |
| o-coumaric | ND | 4.4 | 2.5 | 0.28 |
| acid | | | | |
| p-coumaric | ND | ND | ND | ND |
| acid | | | | |
| procyanidin | 7,670 | 15,000 | 8,910 | 45.0 |

ND = Not Detected
Extract A = hot extract prepared with 50% ethanol and 50% water and macerated for 3 days, lyophilized.
Extract B = Cold extract prepared with 75% ethanol and 25% water and macerated for 3 days, lyophilized.
Extract C = Cold extract prepared with 85% ethanol and 15% water and macerated for 3 days, lyophilized.
Extract D = hot extract prepared with 50% ethanol and 50% water and macerated for 7 days, lyophilized.

The extracts produced with milling/extraction and spray drying were analyzed and the results are shown in table 6.

TABLE 6
Components in Industrial Process Extracts E, F, G:

Concentration (μg/g)

| Compound | Extract E | Extract F | Extract G |
|---|---|---|---|
| Rutin | 47.60 | 46.97 | 47.27 |
| Hispidulin | 25.74 | 30.33 | 30.13 |
| Ferulic acid | 13.90 | 28.75 | 33.91 |
| Unknown ferulic acid | 135.80 | 127.23 | 130.34 |
| Vanillic acid | 7.13 | 23.08 | 33.87 |
| Unknown vanillic acid | 81.58 | 77.03 | 84.47 |
| p-Coumaric Acid | 155.97 | 181.88 | 176.83 |
| Quercetin | 2.15 | 5.24 | 7.67 |
| Naringenin | 7.34 | 10.60 | 9.69 |
| Epicatechin + Catechin | 21,240.00 | 21,910.00 | 22,410.00 |
| procyanidin | 13,090.00 | 16,670.00 | 18,410.00 |

Extracts E, F and G hot extract prepared with 50% ethanol and 50% water and extracted for 2 hours in reactor at 40° C., spray dried

An analysis of an extract produced with maceration and lyophilization aged approximately 10 months (Extract H), and a mixture of extracts produced with maceration and lyophilization aged approximately 3 to 5 years is shown in Table 7 (Extract J). The similar data obtained for Extract H and Extract J indicates that the extracts are relatively stable over time, as well as the reproducibility of the analytical method.

TABLE 7
Stability And Reproducibility

Concentration (μg/g)

| | Extract H | Extract J (mixture) |
|---|---|---|
| Rutin | 61.45 | 81.42 |
| Hispidulin | 20.71 | 31.84 |
| Ferulic acid | Not detected | 36.75 |
| Unknown ferulic acid | 425.77 | 293.61 |
| Vanilic acid | 5.84 | 8.41 |
| Unknown vanillic acid | 95.28 | 87.69 |
| p-Coumaric Acid | 175.50 | 129.83 |
| Quercetin | 2.90 | 3.62 |
| Naringenin | 6.49 | 9.11 |
| Epicatechin + Catechin | 19,550.00 | 11,540.00 |
| procyanidin | 13,410.00 | 8,070.00 |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. As used herein, the term “about” used with a stated value includes numbers 10% above and below the stated value. Unless otherwise stated, a “boiling” temperature or “boiling point” refers to a temperature at which a substance boils at about one atmosphere of pressure. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1.-52. (canceled)

53. A method of preparing an Euterpe oleracea extract, the method comprising:
(a) contacting Euterpe oleracea material with a first solvent at a first temperature below boiling to produce a first wet mixture, and then milling the first wet mixture to obtain a second mixture; or milling Euterpe oleracea material to produce a first dry mixture, and then contacting the first dry mixture with a first solvent at a first temperature below boiling to obtain a second mixture;
(b) contacting the second mixture with an extractor liquid to obtain a crude extract;
(c) filtering the crude extract to obtain a purified extract; and
(d) drying the purified extract to produce the Euterpe oleracea extract using a drying process selected from the group consisting of lyophilization and spray drying;
wherein there is no more than one step that includes heating to at least a boiling point at about one atmosphere, other than the spray drying step.

54. A method of preparing an Euterpe oleracea extract, the method comprising:
(a) contacting Euterpe oleracea material with a first solvent at a first temperature below boiling to produce a first wet mixture, and milling the first wet mixture to obtain a second mixture;
(b) contacting the second mixture with an extractor liquid to obtain a crude extract;
(c) filtering the crude extract to obtain a purified extract; and
(d) lyophilizing the purified extract to produce the Euterpe oleracea extract;
wherein there is no more than one step that includes heating to at least a boiling point at about one atmosphere.

55. The method of claim 53, wherein, in step (a), the first solvent is water, and the first temperature is about 20° C. to about 30° C.

56. The method of claim 53, wherein the Euterpe oleracea material is washed in running water and dried at a temperature between about 20° C. to about 30° C. prior to step (a).

57. The method of claim 53, further comprising wherein in step (a) the second mixture is heated to boiling after milling.

58. The method of claim 53, wherein there is no step that includes heating to at least the boiling point at one atmosphere.

59. The method of claim 58, wherein there is no step that includes heating at a temperature greater than 60° C., greater than 50° C., greater than 40° C., or greater than 30° C.

60. The method of claim 53, wherein during or after milling the first mixture, the milled first mixture is passed through a mesh of 4-16 mm, or about 8 mm, to obtain the second mixture.

61. The method of claim 53, wherein, in step (b), the extractor liquid comprises ethanol.

62. The method of claim 53, comprising maintaining the second mixture contacted with an extractor liquid at a temperature of about 1° C. to about 5° C. for a period of about 1 day to about 10 days.

63. The method of claim 53, wherein step (b) is performed in a reactor for less than 24 hours.

64. The method of claim 63, wherein step (b) is performed under substantially constant agitation and at a temperature of 30-60° C., 35-55° C., about 40° C., or about 50° C.

65. The method of claim 53, wherein in step (b), a ratio of particulate material to the extractor liquid is about 1:6 to about 6:1 weight/volume.

66. The method of claim 53, wherein in step (b), a ratio of particulate material to the extractor liquid is about 1:4 to about 4:1 weight/volume.

67. The method of claim 53, wherein in step (b), a ratio of particulate material to the extractor liquid is from about 1:2 to about 1:10, or about 1:4.

68. The method of claim 53, wherein the extractor liquid comprises ethanol and the first solvent comprises water.

69. The method of claim 68, wherein a percent (v/v) of ethanol to water in step (b) is 75%-85% ethanol and 15-25% water.

70. The method of claim 68, wherein a ratio of ethanol to water (v/v) in step (b) is about 0.1:1 to about 1:1.

71. The method of claim 53, wherein, in step (c), filtering the crude extract comprises:
(i) passing the crude extract through a first filter, wherein the first filter is comprised of a voile-type fabric; and
(ii) passing the crude extract through a second filter, wherein the second filter has a basis weight of about 50-80.0 g/m2.

72. The method of claim 71, wherein the first filter comprises paper.

73. The method of claim 71, wherein the second filter comprises paper of weight from about 50 g/m2 to about 80 g/m2.

74. The method of claim 53, wherein, in step (d), lyophilizing the purified extract comprises:
(i) removing the extractor liquid from the purified extract via rotary evaporation to obtain a concentrated extract; and
(ii) lyophilizing the concentrated extract to produce the Euterpe oleracea extract.

75. The method of claim 74, wherein removing the extractor liquid from the crude extract via rotary evaporation is conducted at a temperature of about 30° C. to about 70° C.

76. The method of claim 74, wherein lyophilization of the concentrated extract is conducted at a temperature of about −35° C. to about −55° C. and a pressure of about 200 μm Hg to about 300 μm Hg.

77. The method of claim 53, further comprising the steps of heating the second mixture to about 90 to 110° C.; and cooling the second mixture to a temperature between about 20° C. to about 30° C. after contacting the second mixture with an extractor liquid and prior to filtering the crude extract.

78. The method of claim 77, wherein heating the second mixture to 90 to 110° C. further comprises holding the second mixture at 90 to 110° C. for about 10 minutes.

79. The method of claim 78, wherein the second mixture is cooled to a temperature of about 20° C.

80. The method of claim 53, wherein, in step (d), spray drying the purified extract comprises
(i) removing the extractor liquid from the purified extract via rotary evaporation to obtain a purified concentrated extract; and
(ii) spray drying the concentrated extract to produce the Euterpe oleracea extract.

81. The method of claim 80, wherein excipients are added to the concentrated purified extract before spray drying in an amount of 40-60% w/v based on a volume of the concentrated purified extract prior to spray drying.

82. The method of claim 81, wherein the excipients comprise maltodextrin and silicon dioxide.

83. The method of claim 80, wherein final temperature during spray drying is 85-105° C.

84. The method of claim 53, wherein the Euterpe oleracea extract has a maximum water content of 6%.

85. The method of claim 53, wherein the Euterpe oleracea extract comprises one or more phenolic compounds selected from the group consisting of vanillic acid, isovanillic acid, ferulic acid, isoferulic acid, hispidulin, rutin, naringenin, quercetin, p-coumaric acid, o-coumaric acid, a procyanidin, epicatechin, catechin, and combinations thereof.

86. An Euterpe oleracea extract composition prepared according to claim 53, comprising:
about 0.5 μg/g to about 50 μg/g vanillic acid;
about 5 μg/g to about 100 μg/g compound of unknown identity, with the same molecular mass of vanillic acid;
about 2 μg/g to about 120 μg/g ferulic acid;
about 5 μg/g to about 200 μg/g compound of unknown identity with the same molecular mass of ferulic acid;
about 0 μg/g to about 250 μg/g p-coumaric acid;
about 2 μg/g to about 80 μg/g hispidulin;
about 10 μg/g to about 70 μg/g rutin;
about 0 mg/g to about 20 mg/g procyanidin;
about 0.5 mg/g to about 30 mg/g epicatechin and catechin;
about 0 μg/g to about 25 μg/g naringenin; and
about 0 μg/g to about 10 μg/g quercetin.
wherein the composition is stable for about 16 weeks at a temperature of from about 20 to about 30° C. as measured by HPLC.

87. An Euterpe oleracea extract composition prepared according to claim 53, comprising:
about 5 μg/g to about 50 μg/g vanillic acid;
about 50 μg/g to about 100 μg/g compound of unknown identity, with the same molecular mass of vanillic acid;
about 10 μg/g to about 50 μg/g ferulic acid;
about 100 μg/g to about 200 μg/g compound of unknown identity with the same molecular mass of ferulic acid;
about 100 μg/g to about 250 μg/g p-coumaric acid;
about 10 μg/g to about 50 μg/g hispidulin;
about 30 μg/g to about 60 μg/g rutin;
about 10 mg/g to about 20 mg/g procyanidin;
about 15 mg/g to about 30 mg/g epicatechin and catechin;
about 5 μg/g to about 15 μg/g naringenin; and
about 0 μg/g to about 10 μg/g quercetin.
wherein the composition is stable for about 16 weeks at a temperature of from about 20 to about 30° C. as measured by HPLC.

88. An Euterpe oleracea extract composition prepared according to claim 53, comprising:
about 1 μg/g to about 4 μg/g vanillic acid;
about 8 μg/g to about 46 μg/g compound of unknown identity, with the same molecular mass of vanillic acid;
about 2.5 μg/g to about 45 μg/g ferulic acid;
about 7 μg/g to about 105 μg/g compound of unknown identity with the same molecular mass of ferulic acid;
about 0 μg/g to about 15 μg/g p-coumaric acid;
about 2 μg/g to about 20 μg/g hispidulin;
about 10 μg/g to about 70 μg/g rutin;
about 0 mg/g to about 20 mg/g procyanidin;
about 100 μg/g to about 1,000 μg/g epicatechin and catechin;
about 5 μg/g to about 15 μg/g naringenin; and
about 0 μg/g to about 10 μg/g quercetin.
wherein the composition is stable for about 16 weeks at a temperature of from about 20 to about 30° C. as measured by HPLC.
